(19) United States
(12) Patent Application Publication
(10) Pub. No.: US 2020/0109354 A1
(43) Pub. Date: Apr. 9, 2020

(54) POLYPEPTIDES

(71) Applicant: Novozymes A/S, Bagsvaerd (DK)

(72) Inventors: Christian Berg Oehlenschlaeger, Valby (DK); Rebecca Munk Vejborg, Allerod (DK); Dorotea Raventos Segura, Rungsted (DK); Jesper Salomon, Holte (DK)

(73) Assignee: Novozymes A/S, Bagsvaerd (DK)

(21) Appl. No.: 16/499,878

(22) PCT Filed: Apr. 4, 2018
(86) PCT No.: PCT/EP2018/058587

§ 371 Date: Oct. 1, 2019

(30) Foreign Application Priority Data
Apr. 4, 2017 (EP) 17164860.3

(51) Int. Cl. C11D 3/386; C12N 9/24

(52) U.S. Cl. CPC C11D 3/38636; C12N 9/2497; C12Y 302/01076; C12Y 302/01037; C12Y 302/02027

(57) ABSTRACT

The present invention relates to polypeptides comprising a GH39 glycosyl hydrolase domain and polynucleotides encoding the polypeptides. The invention further relates to compositions comprising such polypeptides such as cleaning compositions, use of polypeptides comprising the GH39 domain in cleaning processes. The invention further relates to nucleic acid constructs, vectors, and host cells comprising the polynucleotides as well as methods of producing and using the polypeptides.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to polypeptides comprising a GH39 glycosyl hydrolase domain and polynucleotides encoding the polypeptides. The invention further relates to compositions comprising such polypeptides such as cleaning compositions, use of polypeptides comprising the GH39 domain in cleaning processes and/or use of polypeptides comprising the GH39 domain for deep cleaning of biofilm soiling, methods for removal or reduction of biofilm related soiling. The invention further relates to nucleic acid constructs, vectors, and host cells comprising the polynucleotides as well as methods of producing and using the polypeptides.

### Description of the Related Art

Enzymes have been used in detergents for decades. Usually a cocktail of various enzymes is added to detergent compositions. The enzyme cocktail often comprises various enzymes, wherein each enzyme targets it specific substrate e.g. amylases are active towards starch stains, proteases on protein stains and so forth. Textiles and surfaces such as laundry and dishes becomes soiled with many different types of soiling. The soiling may compose of proteins, grease, starch etc. One type of soiling comes from organic matter such as biofilm the presence of biofilm provides several disadvantages. Biofilm comprises an extracellular polymeric matrix, composed of polysaccharides, extracellular DNA (eDNA), and proteins. The extracellular polymeric matrix may be sticky or glueing, which when present on textile, give rise to redeposition or backstaining of soil resulting in a greying of the textile. Another drawback is that malodor may be trapped within the organic structure. Organic matter such as biofilm is therefore not desirable in textiles and surfaces associated with cleaning such as washing machines etc. As organic soiling is a complex mixture of polysaccharides, proteins, DNA etc. there is a need for enzymes which effectively prevent, remove or reduce components of such soiling e.g. polysaccharides of components hereof on items such of fabrics. The object of the present invention is to provide enzymes, which are compatible with cleaning compositions e.g. detergents and which effectively reduce polysaccharides in organic soiling.

### SUMMARY OF THE INVENTION

The present invention provides polypeptides with hydrolase activity, wherein the polypeptides comprise the CAZY database domain GH39 (GH, CAZY database http://www.cazy.org/ (Coutinho & Henrissat, 1999). The domain is a functional domain providing hydrolytic activity to the polypeptide. The invention further provides detergent compositions comprising polypeptides comprising the GH39 domain and the use of such polypeptides for deep cleaning in cleaning processes. The polypeptides of the present invention comprising the GH39 domain have beneficial properties such as deep cleaning in cleaning processes. Cleaning processes include laundry and dish wash. The present invention relates to glycosyl hydrolases in particular GH39 glycosyl hydrolases. One aspect of the invention relates to polypeptides comprising at least one glycosyl hydrolase domain, preferably a GH39 domain. In particular, the invention relates to polypeptides selected from the group consisting of:
(a) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 3;
(b) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 6;
(c) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 9;
(d) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 12;
(e) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 15;
(f) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 18;
(g) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 21;
(h) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 24;
(i) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 27;
(j) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 30;
(k) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 33;
(l) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 36;
(m) a variant of the polypeptide selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33 and SEQ ID NO 36, wherein the variant has hydrolytic activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19 or 20 positions;
(n) a polypeptide comprising the polypeptide of (a) to (I) and a N-terminal and/or C-terminal His-tag and/or HQ-tag;
(o) a polypeptide comprising the polypeptide of (a) to (I) and a N-terminal and/or C-terminal extension of between 1 and 10 amino acids;
(p) a fragment of the polypeptide of (a) to (I) having hydrolytic activity and having at least 90% of the length of the mature polypeptide; and
(q) a polypeptide comprising any of the motifs [A/G/S]XHPY (SEQ ID NO 37) [I/V/L/F/M][Y/W/F]X[T/S]EXG (SEQ ID NO 338), [D/G/I/V]XXX[E/Q][I/L/V]WNE[P/Q/W/F] (SEQ ID NO 39) or [ANTV]WQVW (SEQ ID NO 40).

The invention further relates to a cleaning composition e.g. a detergent composition, a ADW composition, a laundry composition, comprising a polypeptide according to the invention. The invention further relates to use of a polypeptide according to the invention for deep cleaning of an item, such as textile e.g. fabric. The invention further relates to the use of a polypeptide according to the invention,

(i) for preventing, reducing or removing stickiness of the item;

(ii) for pretreating stains on the item;

(iii) for preventing, reducing or removing redeposition of soil during a wash cycle;

(iv) for preventing, reducing or removing adherence of soil to the item;

(v) for maintaining or improving whiteness of the item;

(vi) for preventing, reducing or removing malodor from the item,
wherein the item is a textile.
   The invention also relates to a method for laundering an item comprising the steps of:

a. Exposing an item to a wash liquor comprising a polypeptide according to the invention or a cleaning composition comprising a polypeptide according to the invention;

b. Completing at least one wash cycle; and

c. Optionally rinsing the item,

wherein the item is a textile.

The invention further relates to a polynucleotide encoding the polypeptide of the invention. A nucleic acid construct or expression vector comprising a polynucleotide encoding a polypeptide of the invention, which is operably linked to one or more control sequences that direct the production of the polypeptide in an expression host. The invention further relates to a recombinant host cell comprising a polynucleotide encoding a polypeptide of the invention, which is operably linked to one or more control sequences that direct the production of the polypeptide, wherein the method may further comprise cultivating a cell, which in its wild-type form produces the polypeptide, under conditions conducive for production of the polypeptide and optionally recovering the polypeptide.

### Overview of Sequences

SEQ ID NO 1 DNA encoding full length polypeptide from Pseudomonas fluorescens
SEQ ID NO 2 polypeptide derived from SEQ ID NO 1
SEQ ID NO 3 mature polypeptide obtained from Pseudomonas fluorescens
SEQ ID NO 4 DNA encoding full length polypeptide from Pseudomonas sp-62165
SEQ ID NO 5 polypeptide derived from SEQ ID NO 4
SEQ ID NO 6 mature polypeptide obtained from Pseudomonas sp-62165
SEQ ID NO 7 DNA encoding full length polypeptide from Luteolibacter sp-62326
SEQ ID NO 8 polypeptide derived from SEQ ID NO 7
SEQ ID NO 9 mature polypeptide obtained from Luteolibacter sp-62326
SEQ ID NO 10 DNA encoding full length polypeptide from Pseudomonas sp-62430
SEQ ID NO 11 polypeptide derived from SEQ ID NO 10
SEQ ID NO 12 mature polypeptide obtained from Pseudomonas sp-62430
SEQ ID NO 13 DNA encoding full length polypeptide from Pseudomonas frederiksbergensis
SEQ ID NO 14 polypeptide derived from SEQ ID NO 13
SEQ ID NO 15 mature polypeptide obtained from Pseudomonas frederiksbergensis
SEQ ID NO 16 DNA encoding full length polypeptide from Rhodococcus globerulus
SEQ ID NO 17 polypeptide derived from SEQ ID NO 16
SEQ ID NO 18 mature polypeptide obtained from Rhodococcus globerulus
SEQ ID NO 19 DNA encoding full length polypeptide from Paenibacillus daejeonensis
SEQ ID NO 20 polypeptide derived from SEQ ID NO 19
SEQ ID NO 21 mature polypeptide obtained from Paenibacillus daejeonensis
SEQ ID NO 22 DNA encoding full length polypeptide from Pseudomonas sp-62168
SEQ ID NO 23 polypeptide derived from SEQ ID NO 22
SEQ ID NO 24 mature polypeptide obtained from Pseudomonas sp-62168
SEQ ID NO 25 DNA encoding full length polypeptide from Dyella sp-62115
SEQ ID NO 26 polypeptide derived from SEQ ID NO 25
SEQ ID NO 27 mature polypeptide obtained from Dyella sp-62115
SEQ ID NO 28 DNA encoding full length polypeptide from Pseudomonas fulva
SEQ ID NO 29 polypeptide derived from SEQ ID NO 28
SEQ ID NO 30 mature polypeptide obtained from Pseudomonas fulva
SEQ ID NO 31 DNA encoding full length polypeptide from Rahnella sp-62576
SEQ ID NO 32 polypeptide derived from SEQ ID NO 31
SEQ ID NO 33 mature polypeptide obtained from Rahnella sp-62576
SEQ ID NO 34 DNA encoding full length polypeptide from Pseudomonas aeruginosa
SEQ ID NO 35 polypeptide derived from SEQ ID NO 34
SEQ ID NO 36 mature polypeptide obtained from Pseudomonas aeruginosa
SEQ ID NO 37 conservative motif [A/G/S]XHPY
SEQ ID NO 38 conservative motif [I/V/L/F/M][Y/W/F]X[T/S]EXG
SEQ ID NO 39 conservative motif [D/G/I/V]XXX[E/Q][I/L/V]WNE[P/Q/W/F]
SEQ ID NO 40 conservative motif [ANTV]WQVW

### SEQ ID NO 41 MKKPLGKIVASTALLISVAFSSSIASA

### SEQ ID NO 42 HHHHHHPR

### Definitions

Activity: The present inventions relates to glycosyl hydrolases (EC 3.2.1.-), which are a widespread group of enzymes that hydrolyse the glycosidic bond between two or more carbohydrates or between a carbohydrate and a non-carbohydrate moiety. A classification of glycoside hydrolases in families based on amino acid sequence similarities has been proposed. The polypeptides of the invention comprise at least one glycosyl hydrolase domain and are in the present context defined as glycosyl hydrolases. Thus, polypeptides of the invention hydrolyse glycosidic bonds and the polypeptides of the invention have hydrolytic activity. The glycosyl hydrolase domain comprised in the polypeptide of the invention may be classified as a GH39 domain (PF) and in particular as belonging to the HPY clade and in a preferred embodiment the polypeptides of the invention have hydrolytic (EC 3.2.1.) activity (http://wwvv.cazy.org/). The GH39 glycoside hydrolase family contains two known enzyme activities: β-xylosidase and α-L-iduronidase. Both enzyme activities cleave equatorial glycosidic bonds. The most highly conserved regions in these enzymes located are in their N-terminal sections, Henrissat B, Callebaut I, Mornon J P, Fabrega S, Lehn P, Davies G (1995). “Conserved catalytic machinery and the prediction of a common fold for several families of glycosyl hydrolases”. Proc. Natl. Acad. Sci. U.S.A. 92 (15): 7090-7094.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Biofilm: A biofilm is organic matter produced by any group of microorganisms in which cells stick to each other or stick to a surface, such as a textile, dishware or hard surface or another kind of surface. These adherent cells are frequently embedded within a self-produced matrix of extracellular polymeric substance (EPS). Biofilm EPS is a polymeric conglomeration generally composed of extracellular DNA, proteins, and polysaccharides. Biofilms may form on living or non-living surfaces. The microbial cells growing in a biofilm are physiologically distinct from planktonic cells of the same organism, which, by contrast, are single-cells that may float or swim in a liquid medium. Bacteria living in a biofilm usually have significantly different properties from planktonic bacteria of the same species, as the dense and protected environment of the film allows them to cooperate and interact in various ways. One benefit of this environment for the microorganisms is increased resistance to detergents and antibiotics, as the dense extracellular matrix and the outer layer of cells protect the interior of the community. On laundry biofilm or EPS producing bacteria can be found among the following species: Acinetobacter sp., Aeromicrobium sp., Brevundimonas sp., Microbacterium sp., Micrococcus luteus, Pseudomonas sp., Staphylococcus epidermidis, and Stenotrophomonas sp. In one aspect, the biofilm producing strain is Pseudomonas. In one aspect, the EPS producing strain is Pseudomonas aeruginosa, Pseudomonas alcaliphila or Pseudomonas fluorescens. In one embodiment, the biofilm is caused by microorganisms or group of microorganisms which produce Psl. In another embodiment, the biofilm produce a polysaccharide that is degradable by the GH39 glycosyl hydrolases of the invention. The biofilm that may be formed on the surface e.g. such as textiles may be caused by any microorganism or group of microorganisms that forms Psl-dependent biofilm including but not limited to; Acinetobacter sp., Aeromicrobium sp., Brevundimonas sp., Microbacterium sp., Micrococcus luteus, Staphylococcus epidermidis, Staphylococcus aureus, Pseudomonas sp., Pseudomonas aeruginosa, Pseudomonas alcaliphila, Pseudomonas fluorescens, Stenotrophomonas sp., Paraburkholderia, Burkolderia sp., Candida sp., Bordetella pertussis Yersinia pestis, Escherichia coli and Aspergillus sp.

Catalytic domain: The term “catalytic domain” means the region of an enzyme containing the catalytic machinery of the enzyme.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for introducing specific restriction sites facilitating ligation of the control sequences with the coding region of the polynucleotide encoding a polypeptide.

Deep cleaning: The term “deep cleaning” means disruption, reduction or removal of organic components such as polysaccharides e.g. psl, proteins, DNA, soil or other components present in organic matter such as biofilm.

Detergent adjunct ingredient: The detergent adjunct ingredient or cleaning component is different to the polypeptides of this invention. The precise nature of these additional adjunct components, and levels of incorporation thereof, will depend on the physical form of the composition and the nature of the operation for which it is to be used. Suitable adjunct materials include, but are not limited to the components described below such as surfactants, builders, flocculating aid, chelating agents, dye transfer inhibitors, enzymes, enzyme stabilizers, enzyme inhibitors, catalytic materials, bleach activators, hydrogen peroxide, sources of hydrogen peroxide, preformed peracids, polymeric agents, clay soil removal/anti-redeposition agents, brighteners, suds suppressors, dyes, perfumes, structure elasticizing agents, fabric softeners, carriers, hydrotropes, builders and co-builders, fabric huing agents, anti-foaming agents, dispersants, processing aids, and/or pigments.

Cleaning composition: The term cleaning composition includes “detergent composition” refers to compositions that find use in the removal of undesired compounds from items to be cleaned, such as textiles. The cleaning or detergent composition may be used to e.g. clean textiles for both household cleaning and industrial cleaning. The terms encompass any materials/compounds selected for the particular type of cleaning composition desired and the form of the product (e.g., liquid, gel, powder, granulate, paste, or spray compositions) and includes, but is not limited to, detergent compositions (e.g., liquid and/or solid laundry detergents and fine fabric detergents; fabric fresheners; fabric softeners; and textile and laundry pre-spotters/pretreatment). In addition to containing the enzyme of the invention, the detergent formulation may contain one or more additional enzymes (such as proteases, amylases, lipases, cutinases, cellulases, endoglucanases, xyloglucanases, pectinases, pectin lyases, xanthanases, peroxidases, haloperoxygenases, catalases and mannanases, or any mixture thereof), and/or detergent adjunct ingredients such as surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxido reductases, bluing agents and fluorescent dyes, antioxidants, and solubilizers.

Enzyme Detergency benefit: The term “enzyme detergency benefit” is defined herein as the advantageous effect an enzyme may add to a detergent compared to the same detergent without the enzyme. Important detergency benefits which can be provided by enzymes are stain removal with no or very little visible soils after washing and/or cleaning, prevention or reduction of redeposition of soils released in the washing process (an effect that also is termed anti-redeposition), restoring fully or partly the whiteness of textiles which originally were white but after repeated use and wash have obtained a greyish or yellowish appearance (an effect that also is termed whitening). Textile care benefits, which are not directly related to catalytic stain removal or prevention of redeposition of soils, are also important for enzyme detergency benefits. Examples of such textile care benefits are prevention or reduction of dye transfer from one fabric to another fabric or another part of the same fabric (an effect that is also termed dye transfer inhibition or anti-backstaining), removal of protruding or broken fibers from a fabric surface to decrease pilling tendencies or remove already existing pills or fuzz (an effect that also is termed anti-pilling), improvement of the fabric-softness, colour clarification of the fabric and removal of particulate soils which are trapped in the fibers of the fabric or garment. Enzymatic bleaching is a further enzyme detergency benefit where the catalytic activity generally is used to catalyze the formation of bleaching components such as hydrogen peroxide or other peroxides.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fragment: The term “fragment” means a polypeptide or a catalytic domain having one or more (e.g., several) amino acids absent from the amino and/or carboxyl terminus of a mature polypeptide or domain; wherein the fragment has activity.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance). An isolated substance may be present in a fermentation broth sample; e.g. a host cell may be genetically modified to express the polypeptide of the invention. The fermentation broth from that host cell will comprise the isolated polypeptide.

Improved wash performance: The term “improved wash performance” is defined herein as an enzyme displaying an increased wash performance in a detergent composition relative to the wash performance of same detergent composition without the enzyme e.g. by increased stain removal or less re-deposition. The term “improved wash performance” includes wash performance in laundry.

Laundering: The term “laundering” relates to both household laundering and industrial laundering and means the process of treating textiles with a solution containing a cleaning or detergent composition of the present invention. The laundering process can for example be carried out using e.g. a household or an industrial washing machine or can be carried out by hand.

Malodor: By the term “malodor” is meant an odor which is not desired on clean items. The cleaned item should smell fresh and clean without malodors adhered to the item. One example of malodor is compounds with an unpleasant smell, which may be produced by microorganisms. Another example is unpleasant smells can be sweat or body odor adhered to an item which has been in contact with human or animal. Another example of malodor can be the odor from spices, which sticks to items for example curry or other exotic spices which smells strongly.

Mature polypeptide: The term “mature polypeptide” means a polypeptide in its final form following translation and any post-translational modifications, such as N-terminal processing, C-terminal truncation, glycosylation, phosphorylation, etc. In some aspects, the mature polypeptide is amino acids 1 to 412 of SEQ ID NO 2 and amino acids −30 to −1 of SEQ ID NO 2 is a signal peptide. In some aspects, the mature polypeptide is the amino acid sequence shown in SEQ ID NO 3. In some aspects, the mature polypeptide is amino acids 1 to 411 of SEQ ID NO 5 and amino acids −30 to −1 of SEQ ID NO 5 is a signal peptide. In some aspects, the mature polypeptide is the amino acid sequence shown in SEQ ID NO 6. In some aspects, the mature polypeptide is amino acids 1 to 663 of SEQ ID NO 8 and amino acids −29 to −1 of SEQ ID NO 8 is a signal peptide. In some aspects, the mature polypeptide is the amino acid sequence having SEQ ID NO 9. In some aspects, the mature polypeptide is amino acids 1 to 414 of SEQ ID NO 11 and amino acids −30 to −1 of SEQ ID NO 11 is a signal peptide. In some aspects, the mature polypeptide is the amino acid sequence having SEQ ID NO 12. In some aspects, the mature polypeptide is amino acids 1 to 413 of SEQ ID NO 14 and amino acids −29 to −1 of SEQ ID NO 14 is a signal peptide. In some aspects, the mature polypeptide is the amino acid sequence having SEQ ID NO 15. In some aspects, the mature polypeptide is amino acids 1 to 341 of SEQ ID NO 17 and amino acids −23 to −1 of SEQ ID NO 17 is a signal peptide. In some aspects, the mature polypeptide is the amino acid sequence having SEQ ID NO 18. In some aspects, the mature polypeptide is amino acids 1 to 450 of SEQ ID NO 20 and amino acids −28 to −1 of SEQ ID NO 20 is a signal peptide. In some aspects, the mature polypeptide is the amino acid sequence having SEQ ID NO 21. In some aspects, the mature polypeptide is amino acids 1 to 412 of SEQ ID NO 23 and amino acids −29 to −1 of SEQ ID NO 23 is a signal peptide. In some aspects, the mature polypeptide is the amino acid sequence having SEQ ID NO 24. In some aspects, the mature polypeptide is amino acids 1 to 276 of SEQ ID NO 26 and amino acids −22 to −1 of SEQ ID NO 26 is a signal peptide. In some aspects, the mature polypeptide is the amino acid sequence having SEQ ID NO 27. In some aspects, the mature polypeptide is amino acids 1 to 413 of SEQ ID NO 29 and amino acids −22 to −1 of SEQ ID NO 29 is a signal peptide. In some aspects, the mature polypeptide is the amino acid sequence having SEQ ID NO 30. In some aspects, the mature polypeptide is amino acids 1 to 323 of SEQ ID NO 32 and amino acids −22 to −1 of SEQ ID NO 32 is a signal peptide. In some aspects, the mature polypeptide is the amino acid sequence having SEQ ID NO 33. In some aspects, the mature polypeptide is amino acids 1 to 412 of SEQ ID NO 35. In some aspects, the mature polypeptide is the amino acid sequence having SEQ ID NO 36. It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide having activity. In one aspect, the mature polypeptide coding sequence is nucleotides 91 to 1326 of SEQ ID NO 1 and nucleotides 1 to 90 of SEQ ID NO 1 encodes a signal peptide. In one aspect, the mature polypeptide coding sequence is nucleotides 91 to 1323 of SEQ ID NO 4 and nucleotides 1 to 90 of SEQ ID NO 4 encodes a signal peptide. In one aspect, the mature polypeptide coding sequence is nucleotides 88 to 2076 of SEQ ID NO 7 and nucleotides 1 to 87 of SEQ ID NO 7 encodes a signal peptide. In one aspect, the mature polypeptide coding sequence is nucleotides 91 to 1332 of SEQ ID NO 10 and nucleotides 1 to 90 of SEQ ID NO 10 encodes a signal peptide. In one aspect, the mature polypeptide coding sequence is nucleotides 88 to 1326 of SEQ ID NO 13 and nucleotides 1 to 87 of SEQ ID NO 13 encodes a signal peptide. In one aspect, the mature polypeptide coding sequence is nucleotides 70 to 1092 of SEQ ID NO 16 and nucleotides 1 to 69 of SEQ ID NO 16 encodes a signal peptide. In one aspect, the mature polypeptide coding sequence is nucleotides 85 to 1434 of SEQ ID NO 19 and nucleotides 1 to 84 of SEQ ID NO 19 encodes a signal peptide. In one aspect, the mature polypeptide coding sequence is nucleotides 88 to 1323 of SEQ ID NO 22 and nucleotides 1 to 87 of SEQ ID NO 22 encodes a signal peptide. In one aspect, the mature polypeptide coding sequence is nucleotides 67 to 894 of SEQ ID NO 25 and nucleotides 1 to 66 of SEQ ID NO 25 encodes a signal peptide. In one aspect, the mature polypeptide coding sequence is nucleotides 67 to 1305 of SEQ ID NO 28 and nucleotides 1 to 66 of SEQ ID NO 28 encodes a signal peptide. In one aspect, the mature polypeptide coding sequence is nucleotides 67 to 1035 of SEQ ID NO 31 and nucleotides 1 to 66 of SEQ ID NO 31 encodes a signal peptide. In one aspect, the mature polypeptide coding sequence is nucleotides 1 to 1236 of SEQ ID NO 34.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Nomenclature: For purposes of the present invention, the nomenclature [E/Q] means that the amino acid at this position may be a glutamic acid (Glu, E) or a glutamine (Gln, Q). Likewise, the nomenclature [V/G/A/I] means that the amino acid at this position may be a valine (Val, V), glycine (Gly, G), alanine (Ala, A) or isoleucine (Ile, I), and so forth for other combinations as described herein. Unless otherwise limited further, the amino acid X is defined such that it may be any of the 20 natural amino acids.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

Variant: The term “variant” means a polypeptide having hydrolytic activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position.

### DETAILED DESCRIPTION OF THE INVENTION

Various enzymes are applied in cleaning processes each targeting specific types of soiling such as protein, starch and grease soiling. Very effective often modified enzymes are standard ingredients in detergents for laundry and dish wash. The effectiveness of these commercial enzymes provides detergents which removes much of the soiling. However, organic matters such as EPS (extracellular polymeric substance) comprised in much biofilm constitute a challenging type of soiling due to the complex nature of such organic matters. None of the commercially available detergents effectively remove or reduce EPS related soiling. Biofilm is produced by a group of microorganisms in which cells stick to each other or stick to a surface, such as a textile, dishware or hard surface or another kind of surface. These adherent cells are frequently embedded within a self-produced matrix of extracellular polymeric substance (EPS), which constitute 50% to 90% of the biofilm's total organic matter. EPS is mostly composed of polysaccharides (exopolysaccharides) and proteins, but include other macro-molecules such as DNA, lipids and human substances. EPS is the construction material of bacterial settlements and either remain attached to the cell's outer surface, or is secreted into its growth medium. EPS is required for the development and integrity of biofilms produced by a wide variety of bacteria. The inventors have shown that GH39 glycosyl hydrolase polypeptides of the invention have hydrolytic activity to the exopolysaccharide Psi and thus having the potential to reduce or remove components of EPS and thus reduce or remove EPS related soiling of e.g. textiles. It is well known that polypeptides deriving from organisms may share common structural elements, which can be identified by comparing the primary structures e.g. amino acid sequences and grouping the polypeptides according to sequence homology. However, common structural elements may also be identified by comparing the three-dimensional (3D) structure of various polypeptides. Both approaches have been applied in the present invention.

These approaches identified polypeptides, which derive from organisms from divergent taxonomic groups but share structural elements common for the identified group.

The polypeptides of the invention comprise a GH39 domain and the polypeptides are homologues of PslG enzymes, which are proteins that degrade the exopolysaccharide Psl. The Psi is a pentasaccharide comprising D-glucose, L-rhamnose and D-mannose, which act as a glue in e.g. bacteria surface interactions. PslG is a protein involved in the synthesis of the biofilm matrix exopolysaccharide Psi in Pseudomonas aeruginosa. The polypeptides in GH39 can be separated into distinct sub-clusters, the present inventors have identified one sub-cluster termed HPY, which comprise polypeptides comprising the motif [A/G/S]XHPY (SEQ ID NO 37, former SEQ ID NO 34) situated in positions corresponding to positions 205 to 209 in Pseudomonas fluorescens (SEQ ID NO 3). The H (Histidine) at the position corresponding to position 207 of SEQ ID NO 3 and Y (Tyr) at position 209 are predicted to be involved in substrate binding. Another motif characteristic of HPY domain is [I/V/L/F/M][Y/W/F]X[T/S]EXG (SEQ ID NO 38, former SEQ ID NO 35), corresponding to position 242 to 248 in SEQ ID NO 3. Yet a further motif of the HPY domain identified by the inventors is [D/G/I/V]XXX[E/Q][I/L/V]WNE[P/Q/W/F] (SEQ ID NO 39, former SEQ ID NO 36), corresponding to position 127 to 136 in SEQ ID NO 3, where N (Asn) and E (Glu) at positions 134 and 135 are predicted to be involved in substrate binding. In one embodiment of the invention the polypeptides of the present invention share the common motif [A/G/S]XHPY] (SEQ ID NO 37). In one embodiment of the invention the polypeptides of the present invention share the common motif [I/V/L/F/M][Y/W/F]X[T/S]EXG (SEQ ID NO: 38). In one embodiment of the invention the polypeptides of the present invention share the common motif [D/G/I/V]XXX[E/Q][I/L/V]WNE[P/Q/W/F] (SEQ ID NO 39). In one embodiment the polypeptides of the invention comprises one or more, or even all three of the motif(s) [A/G/S]XHPY (SEQ ID NO 37) or [I/V/L/F/M][Y/W/F]X[T/S]EXG (SEQ ID NO 38) or [D/G/I/V]XXX[E/Q][I/L/V]WNE[P/Q/W/F] (SEQ ID NO 39). Within the HPY domain the present inventors identified a clade termed the WQVW clade, which comprises HPY polypeptides of bacterial origin, having activity on Psl. The polypeptides of the clade comprise the motif [ANTV]WQVW (SEQ ID NO 40, former SEQ ID NO:37), corresponding to pos 129 to 133 of Pseudomonas fluorescens (SEQ ID NO 3). In one embodiment the polypeptides of the invention comprises one or more, or even all of the motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO:40). In one embodiment, the polypeptides of the invention belong to the WQVW clade, comprises the a glycosyl hydrolytic domain and have hydrolytic activity. In one embodiment, the polypeptides of the invention are of bacterial origin (i.e. is obtained from bacteria), have activity on Psi and comprises the motif [ANTV]WQVW (SEQ ID NO: 40). One embodiment relates to a GH39 glycosyl hydrolase comprising one or more, or even all of the motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO: 40). One embodiment relates to a GH39 glycosyl hydrolase comprising two or more, or even all of the motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO: 40). One embodiment relates to a GH39 glycosyl hydrolase comprising three or more, or even all of the motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO: 40). One embodiment relates to a GH39 glycosyl hydrolase comprising all four motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO: 40).

One embodiment of the invention relates a polypeptide comprising the GH39 domain, wherein the polypeptide has hydrolytic activity, and wherein the polypeptide is selected from the group consisting of:
(a) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 3;
(b) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 6;
(c) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 9;
(d) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 12;
(e) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 15;
(f) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 18;
(g) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 21;
(h) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 24;
(i) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 27;
(j) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 30;
(k) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100%% sequence identity to the polypeptide of SEQ ID NO: 33;
(l) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 36;
(m) a variant of the polypeptide selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33 and SEQ ID NO: 36, wherein the variant has hydrolytic activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19 or 20 positions;
(n) a polypeptide comprising the polypeptide of (a) to (I) and a N-terminal and/or C-terminal His-tag and/or HQ-tag;
(o) a polypeptide comprising the polypeptide of (a) to (I) and a N-terminal and/or C-terminal extension of between 1 and 10 amino acids; and
(p) a fragment of the polypeptide of (a) to (I) having hydrolytic activity and having at least 90% of the length of the mature polypeptide
(q) a polypeptide comprising one or more, or even all of the motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO: 40).

One embodiment of the invention relates a polypeptide comprising the GH39 domain, wherein the polypeptide has hydrolytic activity, and wherein the polypeptide is selected from the group consisting of:
(a) a polypeptide having at least 99% or 100% sequence identity to the polypeptide of SEQ ID NO: 3;
(b) a polypeptide having at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 6;
(c) a polypeptide having at least 50%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 9;
(d) a polypeptide having at least 91%, at least 92%, at least 93%, at least 94%, at least 95%, at least 96%, at least 97%, at least 98%, at least 99% or 100% sequence identity to the polypeptide of SEQ ID NO: 12;
(e) a polypeptide having at least 90%, at least 91%, at least 92%, at least 93%, at least 94%, at least 95%, at least 96%, at least 97%, at least 98%, at least 99% or 100% sequence identity to the polypeptide of SEQ ID NO: 15;
(f) a polypeptide having 100% sequence identity, comprising or consisting of the polypeptide of SEQ ID NO: 18;
(g) a polypeptide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 21;
(h) a polypeptide having at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 24;
(i) a polypeptide having at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 27;
(j) a polypeptide having at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 30;
(k) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 33; and
(l) a polypeptide having at least 90%, at least 91%, at least 92%, at least 93%, at least 94%, at least 95%, at least 96%, at least 97%, at least 98%, at least 99% or 100% sequence identity to the polypeptide of SEQ ID NO: 36.

One embodiment relates to a polypeptide comprising one or more, or even all of the motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO: 40), wherein and wherein the polypeptide is selected from the group consisting of:
(a) a polypeptide having at least 99% or 100% sequence identity to the polypeptide of SEQ ID NO: 3;
(b) a polypeptide having at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 6;
(c) a polypeptide having at least 50%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 9;
(d) a polypeptide having at least 91%, at least 92%, at least 93%, at least 94%, at least 95%, at least 96%, at least 97%, at least 98%, at least 99% or 100% sequence identity to the polypeptide of SEQ ID NO: 12;
(e) a polypeptide having at least 90%, at least 91%, at least 92%, at least 93%, at least 94%, at least 95%, at least 96%, at least 97%, at least 98%, at least 99% or 100% sequence identity to the polypeptide of SEQ ID NO: 15;
(f) a polypeptide having 100% sequence identity, comprising or consisting of the polypeptide of SEQ ID NO: 18;
(g) a polypeptide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 21;
(h) a polypeptide having at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 24;
(i) a polypeptide having at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 27;
(j) a polypeptide having at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 30;
(k) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 33; and
(l) a polypeptide having at least 90%, at least 91%, at least 92%, at least 93%, at least 94%, at least 95%, at least 96%, at least 97%, at least 98%, at least 99% or 100% sequence identity to the polypeptide of SEQ ID NO: 36.

The polypeptides of the invention have activity to the exopolysaccharide Psi, which is a component of some biofilm matrix. One embodiment of the invention relates to the use of a polypeptide according to the invention for reduction or removal of Psi, wherein in the Psi is comprised in a biofilm. In particular, the polypeptides of the invention have activity in detergents and is useful in cleaning processes such as laundry and/or dish wash e.g. for cleaning e.g. deep cleaning of surfaces such as textiles and hard surfaces. The present disclosure also provides a method for preventing, reduction or removal of Psi containing organic soiling on an item comprising applying at least one polypeptide of the invention to an item and optionally rinse the item. The item is preferably a textile or a hard surface e.g. a non-medical hard surface, such as dish ware. Organic matters such as EPS or components hereof may have glue-like properties and the presence of biofilm on e.g. textiles may result in items or areas on items which are “sticky”. Soil will in general adhere to the sticky areas and such soil has shown difficult to remove by commercially available detergent compositions. Further, when dirty laundry items are washed together with less dirty laundry items the dirt present in the wash liquor tend to stick to the organic matter and e.g. EPS. As a result, the laundry item is more “soiled” after wash than before wash. This is effect may also be termed re-deposition. The polypeptides comprising one or more of the motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO:40) as defined above are useful in reducing or removing re-deposition.

The polypeptides comprising one or more of the motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO:40) as defined above are useful in reducing or removing malodor of items being washed. The inventors have surprisingly found that the polypeptides comprising one or more of the motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO:40) as defined above are useful in reducing or removing laundry associated Psl.

The polypeptides of the present invention are useful in cleaning compositions and are effective in deep cleaning of surfaces such as fabrics. The polypeptides of the present invention are effective in reducing or removing polysaccharide soiling from e.g. organic matter such as EPS. One example of organic matter is biofilm, which is an extracellular matrix produced by various microorganisms. The extracellular polymeric matrix is composed of polysaccharides, extracellular DNA and proteins. Organic matter like biofilm may be sticky or glueing, which when present on textile, may give rise to re-deposition or backstaining of soil resulting in a greying of the textile. Another drawback of organic matter e.g. biofilm is the malodor as various malodor related molecules are often associated with organic matter e.g. biofilm. One aspect of the invention relates to a laundering method for laundering an item comprising the steps of:

a. exposing an item to a wash liquor comprising a polypeptide or a cleaning composition comprising a polypeptide selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO 36 or polypeptides having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity hereto, wherein the polypeptide has hydrolytic activity;

b. completing at least one wash cycle; and

c. optionally rinsing the item,

wherein the item is a textile.

The polypeptides of the invention are therefore useful for prevention, reduction or removal of malodor and for prevention, reduction of re-deposition and improving whiteness.

One embodiment of the invention relates to the use of polypeptide selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO 36 or polypeptides having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity hereto for cleaning e.g. deep cleaning of an item, wherein the item is a textile. One embodiment of the invention relates to the use of polypeptide selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO 36 or polypeptides having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity hereto;

(i) for preventing, reducing or removing stickiness of the item;

(ii) for pretreating stains on the item;

(iii) for preventing, reducing or removing redeposition of soil during a wash cycle;

(iv) for preventing, reducing or removing adherence of soil to the item;

(v) for maintaining or improving whiteness of the item;

(vi) for preventing, reducing or removal malodor from the item,
wherein the item is a textile.

The textile may e.g. be cotton or polyester or a mixture hereof.

One embodiment of the invention relates to a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide shown in SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33 and SEQ ID NO 36.

In some embodiment, the present invention relates to polypeptides having a sequence identity to the mature polypeptide of SEQ ID NO: 2 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 2.

In a particular embodiment, the invention relates to polypeptides having a sequence identity to the mature polypeptide of SEQ ID NO: 5 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 5.

In a particular embodiment, the invention relates to polypeptides having a sequence identity to the mature polypeptide of SEQ ID NO: 8 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 8.

In a particular embodiment, the invention relates to polypeptides having a sequence identity to the mature polypeptide of SEQ ID NO: 11 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 11.

In a particular embodiment, the invention relates to polypeptides having a sequence identity to the mature polypeptide of SEQ ID NO: 14 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 14.

In some embodiment, the present invention relates to polypeptides having a sequence identity to the mature polypeptide of SEQ ID NO: 17 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 17.

In a particular embodiment, the invention relates to polypeptides having a sequence identity to the mature polypeptide of SEQ ID NO: 20 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 20.

In a particular embodiment, the invention relates to polypeptides having a sequence identity to the mature polypeptide of SEQ ID NO: 23 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 23.

In a particular embodiment, the invention relates to polypeptides having a sequence identity to the mature polypeptide of SEQ ID NO: 26 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 26.

In a particular embodiment, the invention relates to polypeptides having a sequence identity to the mature polypeptide of SEQ ID NO: 29 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 29.

In a particular embodiment, the invention relates to polypeptides having a sequence identity to the mature polypeptide of SEQ ID NO: 32 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 32.

In a particular embodiment, the invention relates to polypeptides having a sequence identity to the mature polypeptide of SEQ ID NO: 35 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the hydrolytic activity of the mature polypeptide of SEQ ID NO: 35.

In some embodiment, the polypeptide has been isolated. A polypeptide of the present invention preferably comprises or consists of the amino acid sequence shown in SEQ ID NO: 3 or an allelic variant thereof; or is a fragment thereof having hydrolytic activity. In another aspect, the polypeptide comprises or consists of the mature polypeptide of SEQ ID NO: 2. In another aspect, the polypeptide comprises or consists of amino acids 1 to 412 of SEQ ID NO: 2.

In one embodiment, the polypeptide preferably comprises or consists of the amino acid sequence shown in SEQ ID NO: 3; comprises the amino acid sequence shown in SEQ ID NO: 3 and a N-terminal and/or C-terminal His-tag and/or HQ-tag; comprises the amino acid sequence of SEQ ID NO: 3 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 3.

In some embodiment, the polypeptide has been isolated. A polypeptide of the present invention preferably comprises or consists of the amino acid sequence shown in SEQ ID NO: 6 or an allelic variant thereof; or is a fragment thereof having hydrolytic activity. In another aspect, the polypeptide comprises or consists of the mature polypeptide of SEQ ID NO: 5. In another aspect, the polypeptide comprises or consists of amino acids 1 to 411 of SEQ ID NO: 5.

In one embodiment, the polypeptide preferably comprises or consists of the amino acid sequence shown in SEQ ID NO: 6; comprises the amino acid sequence shown in SEQ ID NO: 6 and a N-terminal and/or C-terminal His-tag and/or HQ-tag; comprises the amino acid sequence of SEQ ID NO: 6 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 6.

In some embodiment, the polypeptide has been isolated. A polypeptide of the present invention preferably comprises or consists of the amino acid sequence shown in SEQ ID NO: 9 or an allelic variant thereof; or is a fragment thereof having hydrolytic activity. In another aspect, the polypeptide comprises or consists of the mature polypeptide of SEQ ID NO: 8. In another aspect, the polypeptide comprises or consists of amino acids 1 to 663 of SEQ ID NO: 8.

In one embodiment, the polypeptide preferably comprises or consists of the amino acid sequence shown in SEQ ID NO: 9; comprises the amino acid sequence shown in SEQ ID NO: 9 and a N-terminal and/or C-terminal His-tag and/or HQ-tag; comprises the amino acid sequence of SEQ ID NO: 9 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 9.

In some embodiment, the polypeptide has been isolated. A polypeptide of the present invention preferably comprises or consists of the amino acid sequence shown in SEQ ID NO: 12 or an allelic variant thereof; or is a fragment thereof having hydrolytic activity. In another aspect, the polypeptide comprises or consists of the mature polypeptide of SEQ ID NO: 11. In another aspect, the polypeptide comprises or consists of amino acids 1 to 414 of SEQ ID NO: 11.

In one embodiment, the polypeptide preferably comprises or consists of the amino acid sequence shown in SEQ ID NO: 12; comprises the amino acid sequence shown in SEQ ID NO: 12 and a N-terminal and/or C-terminal His-tag and/or HQ-tag; comprises the amino acid sequence of SEQ ID NO: 12 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 12.

In some embodiment, the polypeptide has been isolated. A polypeptide of the present invention preferably comprises or consists of the amino acid sequence shown in SEQ ID NO: 15 or an allelic variant thereof; or is a fragment thereof having hydrolytic activity. In another aspect, the polypeptide comprises or consists of the mature polypeptide of SEQ ID NO: 14. In another aspect, the polypeptide comprises or consists of amino acids 1 to 413 of SEQ ID NO: 14.

In one embodiment, the polypeptide preferably comprises or consists of the amino acid sequence shown in SEQ ID NO: 15; comprises the amino acid sequence shown in SEQ ID NO: 15 and a N-terminal and/or C-terminal His-tag and/or HQ-tag; comprises the amino acid sequence of SEQ ID NO: 15 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 15.

In some embodiment, the polypeptide has been isolated. A polypeptide of the present invention preferably comprises or consists of the amino acid sequence shown in SEQ ID NO: 18 or an allelic variant thereof; or is a fragment thereof having hydrolytic activity. In another aspect, the polypeptide comprises or consists of the mature polypeptide of SEQ ID NO: 17. In another aspect, the polypeptide comprises or consists of amino acids 1 to 341 of SEQ ID NO: 17.

In one embodiment, the polypeptide preferably comprises or consists of the amino acid sequence shown in SEQ ID NO: 18; comprises the amino acid sequence shown in SEQ ID NO: 18 and a N-terminal and/or C-terminal His-tag and/or HQ-tag; comprises the amino acid sequence of SEQ ID NO: 18 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 18.

In some embodiment, the polypeptide has been isolated. A polypeptide of the present invention preferably comprises or consists of the amino acid sequence shown in SEQ ID NO: 21 or an allelic variant thereof; or is a fragment thereof having hydrolytic activity. In another aspect, the polypeptide comprises or consists of the mature polypeptide of SEQ ID NO: 20. In another aspect, the polypeptide comprises or consists of amino acids 1 to 450 of SEQ ID NO: 20.

In one embodiment, the polypeptide preferably comprises or consists of the amino acid sequence shown in SEQ ID NO: 21; comprises the amino acid sequence shown in SEQ ID NO: 21 and a N-terminal and/or C-terminal His-tag and/or HQ-tag; comprises the amino acid sequence of SEQ ID NO: 21 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 21.

In some embodiment, the polypeptide has been isolated. A polypeptide of the present invention preferably comprises or consists of the amino acid sequence shown in SEQ ID NO: 24 or an allelic variant thereof; or is a fragment thereof having hydrolytic activity. In another aspect, the polypeptide comprises or consists of the mature polypeptide of SEQ ID NO: 23. In another aspect, the polypeptide comprises or consists of amino acids 1 to 412 of SEQ ID NO: 23.

In one embodiment, the polypeptide preferably comprises or consists of the amino acid sequence shown in SEQ ID NO: 27; comprises the amino acid sequence shown in SEQ ID NO: 27 and a N-terminal and/or C-terminal His-tag and/or HQ-tag; comprises the amino acid sequence of SEQ ID NO: 27 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 27.

In some embodiment, the polypeptide has been isolated. A polypeptide of the present invention preferably comprises or consists of the amino acid sequence shown in SEQ ID NO: 27 or an allelic variant thereof; or is a fragment thereof having hydrolytic activity. In another aspect, the polypeptide comprises or consists of the mature polypeptide of SEQ ID NO: 26. In another aspect, the polypeptide comprises or consists of amino acids 1 to 276 of SEQ ID NO: 26.

In one embodiment, the polypeptide preferably comprises or consists of the amino acid sequence shown in SEQ ID NO: 30; comprises the amino acid sequence shown in SEQ ID NO: 30 and a N-terminal and/or C-terminal His-tag and/or HQ-tag; comprises the amino acid sequence of SEQ ID NO: 30 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 30.

In some embodiment, the polypeptide has been isolated. A polypeptide of the present invention preferably comprises or consists of the amino acid sequence shown in SEQ ID NO: 30 or an allelic variant thereof; or is a fragment thereof having hydrolytic activity. In another aspect, the polypeptide comprises or consists of the mature polypeptide of SEQ ID NO: 29. In another aspect, the polypeptide comprises or consists of amino acids 1 to 413 of SEQ ID NO: 29.

In one embodiment, the polypeptide preferably comprises or consists of the amino acid sequence shown in SEQ ID NO: 33; comprises the amino acid sequence shown in SEQ ID NO: 33 and a N-terminal and/or C-terminal His-tag and/or HQ-tag; comprises the amino acid sequence of SEQ ID NO: 33 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 33.

In some embodiment, the polypeptide has been isolated. A polypeptide of the present invention preferably comprises or consists of the amino acid sequence shown in SEQ ID NO: 33 or an allelic variant thereof; or is a fragment thereof having hydrolytic activity. In another aspect, the polypeptide comprises or consists of the mature polypeptide of SEQ ID NO: 32. In another aspect, the polypeptide comprises or consists of amino acids 1 to 323 of SEQ ID NO: 32.

In one embodiment, the polypeptide preferably comprises or consists of the amino acid sequence shown in SEQ ID NO: 36; comprises the amino acid sequence shown in SEQ ID NO: 36 and a N-terminal and/or C-terminal His-tag and/or HQ-tag; comprises the amino acid sequence of SEQ ID NO: 36 and a N-terminal and/or C-terminal extension of between 1 and 10 amino acids; or is a fragment thereof having hydrolytic activity and having at least 50% such as at least 60%, at least 70%, at least 75%, at least 80%, at least 85%, at least 90%, at least 95%, at least 96%, at least 97%, at least 98% or at least 99% of the length of SEQ ID NO: 36.

In some embodiment, the polypeptide has been isolated. A polypeptide of the present invention preferably comprises or consists of the amino acid sequence shown in SEQ ID NO: 36 or an allelic variant thereof; or is a fragment thereof having hydrolytic activity. In another aspect, the polypeptide comprises or consists of the mature polypeptide of SEQ ID NO: 35. In another aspect, the polypeptide comprises or consists of amino acids 1 to 412 of SEQ ID NO: 35.

In some aspects, the invention relates to a polypeptide which comprises or consists of the amino acid sequence shown in SEQ ID NO 3.

In some aspects, the invention relates to a polypeptide which comprises or consists of the amino acid sequence shown in SEQ ID NO 6.

In some aspects, the invention relates to a polypeptide which comprises or consists of the amino acid sequence shown in SEQ ID NO 9.

In some aspects, the invention relates to a polypeptide which comprises or consists of the amino acid sequence shown in SEQ ID NO 12.

In some aspects, the invention relates to a polypeptide which comprises or consists of the amino acid sequence shown in SEQ ID NO 15.

In some aspects, the invention relates to a polypeptide which comprises or consists of the amino acid sequence shown in SEQ ID NO 18.

In some aspects, the invention relates to a polypeptide which comprises or consists of the amino acid sequence shown in SEQ ID NO 21.

In some aspects, the invention relates to a polypeptide which comprises or consists of the amino acid sequence shown in SEQ ID NO 24.

In some aspects, the invention relates to a polypeptide which comprises or consists of the amino acid sequence shown in SEQ ID NO 27.

In some aspects, the invention relates to a polypeptide which comprises or consists of the amino acid sequence shown in SEQ ID NO 30.

In some aspects, the invention relates to a polypeptide which comprises or consists of the amino acid sequence shown in SEQ ID NO 33.

In some aspects, the invention relates to a polypeptide which comprises or consists of the amino acid sequence shown in SEQ ID NO 36.

In some embodiment, the present invention relates to variants of the mature polypeptide shown in SEQ ID NO: 3 comprising a substitution, deletion, and/or insertion at one or more (e.g., several) positions. In some embodiment, the number of amino acid substitutions, deletions and/or insertions introduced into the mature polypeptide shown in SEQ ID NO: 3 is up to 10, e.g., 1, 2, 3, 4, 5, 6, 7, 8, 9, or 10.

In some embodiment, the present invention relates to variants of the mature polypeptide shown in SEQ ID NO: 6 comprising a substitution, deletion, and/or insertion at one or more (e.g., several) positions. In some embodiment, the number of amino acid substitutions, deletions and/or insertions introduced into the mature polypeptide shown in SEQ ID NO: 6 is up to 10, e.g., 1, 2, 3, 4, 5, 6, 7, 8, 9, or 10.

In some embodiment, the present invention relates to variants of the mature polypeptide shown in SEQ ID NO: 9 comprising a substitution, deletion, and/or insertion at one or more (e.g., several) positions. In some embodiment, the number of amino acid substitutions, deletions and/or insertions introduced into the mature polypeptide shown in SEQ ID NO: 9 is up to 10, e.g., 1, 2, 3, 4, 5, 6, 7, 8, 9, or 10.

In some embodiment, the present invention relates to variants of the mature polypeptide shown in SEQ ID NO: 12 comprising a substitution, deletion, and/or insertion at one or more (e.g., several) positions. In some embodiment, the number of amino acid substitutions, deletions and/or insertions introduced into the mature polypeptide shown in SEQ ID NO: 12 is up to 10, e.g., 1, 2, 3, 4, 5, 6, 7, 8, 9, or 10.

In some embodiment, the present invention relates to variants of the mature polypeptide shown in SEQ ID NO: 15 comprising a substitution, deletion, and/or insertion at one or more (e.g., several) positions. In some embodiment, the number of amino acid substitutions, deletions and/or insertions introduced into the mature polypeptide shown in SEQ ID NO: 15 is up to 10, e.g., 1, 2, 3, 4, 5, 6, 7, 8, 9, or 10.

In some embodiment, the present invention relates to variants of the mature polypeptide shown in SEQ ID NO: 18 comprising a substitution, deletion, and/or insertion at one or more (e.g., several) positions. In some embodiment, the number of amino acid substitutions, deletions and/or insertions introduced into the mature polypeptide shown in SEQ ID NO: 18 is up to 10, e.g., 1, 2, 3, 4, 5, 6, 7, 8, 9, or 10.

In some embodiment, the present invention relates to variants of the mature polypeptide shown in SEQ ID NO: 21 comprising a substitution, deletion, and/or insertion at one or more (e.g., several) positions. In some embodiment, the number of amino acid substitutions, deletions and/or insertions introduced into the mature polypeptide shown in SEQ ID NO: 21 is up to 10, e.g., 1, 2, 3, 4, 5, 6, 7, 8, 9, or 10.

In some embodiment, the present invention relates to variants of the mature polypeptide shown in SEQ ID NO: 24 comprising a substitution, deletion, and/or insertion at one or more (e.g., several) positions. In some embodiment, the number of amino acid substitutions, deletions and/or insertions introduced into the mature polypeptide shown in SEQ ID NO: 24 is up to 10, e.g., 1, 2, 3, 4, 5, 6, 7, 8, 9, or 10.

In some embodiment, the present invention relates to variants of the mature polypeptide shown in SEQ ID NO: 27 comprising a substitution, deletion, and/or insertion at one or more (e.g., several) positions. In some embodiment, the number of amino acid substitutions, deletions and/or insertions introduced into the mature polypeptide shown in SEQ ID NO: 27 is up to 10, e.g., 1, 2, 3, 4, 5, 6, 7, 8, 9, or 10.

In some embodiment, the present invention relates to variants of the mature polypeptide shown in SEQ ID NO: 30 comprising a substitution, deletion, and/or insertion at one or more (e.g., several) positions. In some embodiment, the number of amino acid substitutions, deletions and/or insertions introduced into the mature polypeptide shown in SEQ ID NO: 30 is up to 10, e.g., 1, 2, 3, 4, 5, 6, 7, 8, 9, or 10.

In some embodiment, the present invention relates to variants of the mature polypeptide shown in SEQ ID NO: 33 comprising a substitution, deletion, and/or insertion at one or more (e.g., several) positions. In some embodiment, the number of amino acid substitutions, deletions and/or insertions introduced into the mature polypeptide shown in SEQ ID NO: 33 is up to 10, e.g., 1, 2, 3, 4, 5, 6, 7, 8, 9, or 10.

In some embodiment, the present invention relates to variants of the mature polypeptide shown in SEQ ID NO: 33 comprising a substitution, deletion, and/or insertion at one or more (e.g., several) positions. In some embodiment, the number of amino acid substitutions, deletions and/or insertions introduced into the mature polypeptide shown in SEQ ID NO: 36 is up to 10, e.g., 1, 2, 3, 4, 5, 6, 7, 8, 9, or 10.

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are tested for hydrolytic activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The polypeptide may be a hybrid polypeptide in which a region of one polypeptide is fused at the N-terminus or the C-terminus of a region of another polypeptide.

The polypeptide may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

### Sources of Polypeptides Having Polypeptide Activity

A polypeptide having hydrolytic activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly. In one aspect, the polypeptide is a Pseudomonas polypeptide, e.g., a polypeptide obtained from Pseudomonas sp-62165, Pseudomonas sp-62326, Pseudomonas sp-62430 or Pseudomonas fluorescens, Pseudomonas frederiksbergensis, Pseudomonas aeruginosa or Pseudomonas fulva. In one aspect, the polypeptide is a Luteolibacter polypeptide, e.g., a polypeptide obtained from Luteolibacter sp-62326. In one aspect, the polypeptide is a Rhodococcus polypeptide, e.g., a polypeptide obtained from Rhodococcus globerulus. In one aspect, the polypeptide is a Paenibacillus polypeptide, e.g., a polypeptide obtained from Paenibacillus daejeonensis. In one aspect, the polypeptide is a Dyella polypeptide, e.g., a polypeptide obtained from Dyella sp-62115. In one aspect, the polypeptide is a Rahnella polypeptide, e.g., a polypeptide obtained from Rahnella sp-62576. In one embodiment, the GH39 glycosyl hydrolase i.e. a polypeptide comprising the GH39 domain is bacterial. In one embodiment, the GH39 glycosyl hydrolase i.e. a polypeptide comprising the GH39 domain is derived from Pseudomonas e.g. Pseudomonas sp-62165, Pseudomonas sp-62326,

Pseudomonas sp-62430 or Pseudomonas fluorescens, Pseudomonas frederiksbergensis, Pseudomonas aeruginosa or Pseudomonas fulva. In one embodiment, the GH39 glycosyl hydrolase is obtained from Pseudomonas, preferably Pseudomonas sp-62165, Pseudomonas sp-62326, Pseudomonas sp-62430 or Pseudomonas fluorescens, Pseudomonas frederiksbergensis, Pseudomonas aeruginosa or Pseudomonas fulva, wherein the GH39 glycosyl hydrolase comprising one or more, or even all of the motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO: 40).

In one embodiment, the GH39 glycosyl hydrolase is obtained from Pseudomonas, preferably Pseudomonas sp-62165, Pseudomonas sp-62326, Pseudomonas sp-62430 or Pseudomonas fluorescens, Pseudomonas frederiksbergensis, Pseudomonas aeruginosa or Pseudomonas fulva, wherein the GH39 glycosyl hydrolase is selected from the group consisting of:
(a) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 3;
(b) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 6;
(d) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 12;
(e) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 15;
(h) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 24;
(j) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 30; and
(l) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 36.

In one embodiment, the GH39 glycosyl hydrolase i.e. a polypeptide comprising the GH39 domain is bacterial. In one embodiment, the GH39 glycosyl hydrolase i.e. a polypeptide comprising the GH39 domain is derived from Luteolibacter e.g. Luteolibacter sp. In one embodiment, the GH39 glycosyl hydrolase is obtained from Luteolibacter, wherein the GH39 glycosyl hydrolase comprising one or more, or even all of the motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO: 40).

In one embodiment, the GH39 glycosyl hydrolase is obtained from Luteolibacter, wherein the GH39 glycosyl hydrolase is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 9.

In one embodiment, the GH39 glycosyl hydrolase i.e. a polypeptide comprising the GH39 domain is bacterial. In one embodiment, the GH39 glycosyl hydrolase i.e. a polypeptide comprising the GH39 domain is derived from Rhodococcus e.g. Rhodococcus globerulus. In one embodiment, the GH39 glycosyl hydrolase is obtained from Rhodococcus, preferably Rhodococcus globerulus, wherein the GH39 glycosyl hydrolase comprising one or more, or even all of the motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO: 40).

In one embodiment, the GH39 glycosyl hydrolase is obtained from Rhodococcus, preferably Rhodococcus globerulus, wherein the GH39 glycosyl hydrolase is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 18.

In one embodiment, the GH39 glycosyl hydrolase i.e. a polypeptide comprising the GH39 domain is bacterial. In one embodiment, the GH39 glycosyl hydrolase i.e. a polypeptide comprising the GH39 domain is derived from Paenibacillus e.g. Paenibacillus daejeonensis. In one embodiment, the GH39 glycosyl hydrolase is obtained from Paenibacillus, preferably Paenibacillus daejeonensis, wherein the GH39 glycosyl hydrolase comprising one or more, or even all of the motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO: 40). In one embodiment, the GH39 glycosyl hydrolase is obtained from Paenibacillus, preferably Paenibacillus daejeonensis, wherein the GH39 glycosyl hydrolase is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 21.

In one embodiment, the GH39 glycosyl hydrolase i.e. a polypeptide comprising the GH39 domain is bacterial. In one embodiment, the GH39 glycosyl hydrolase i.e. a polypeptide comprising the GH39 domain is derived from Dyella. In one embodiment, the GH39 glycosyl hydrolase is obtained from Dyella, wherein the GH39 glycosyl hydrolase comprising one or more, or even all of the motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO: 40). In one embodiment, the GH39 glycosyl hydrolase is obtained from Dyella, wherein the GH39 glycosyl hydrolase is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 27.

In one embodiment, the GH39 glycosyl hydrolase i.e. a polypeptide comprising the GH39 domain is bacterial. In one embodiment, the GH39 glycosyl hydrolase i.e. a polypeptide comprising the GH39 domain is derived from Rahnella. In one embodiment, the GH39 glycosyl hydrolase is obtained from Rahnella, wherein the GH39 glycosyl hydrolase comprising one or more, or even all of the motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO: 40). In one embodiment, the GH39 glycosyl hydrolase is obtained from Rahnella, wherein the GH39 glycosyl hydrolase is a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 33.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Polynucleotides

The present invention also relates to polynucleotides encoding a polypeptide of the present invention, as described herein. In some embodiment, the polynucleotide encoding the polypeptide of the present invention has been isolated.

In some embodiment, the present invention relates to a polynucleotide encoding a polypeptide having hydrolytic activity, wherein the polynucleotide having a sequence identity to the mature polypeptide coding sequence of SEQ ID NO: 1 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 4 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 7 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 10 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 13 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 16 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 19 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 22 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 25 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 28 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 31 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

In some embodiment, the present invention relates to a polynucleotide encoding a polypeptide having hydrolytic activity, wherein the polynucleotide having a sequence identity to the mature polypeptide coding sequence of SEQ ID NO: 34 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polynucleotide has been isolated.

The techniques used to isolate or clone a polynucleotide are known in the art and include isolation from genomic DNA or cDNA, or a combination thereof. The cloning of the polynucleotides from genomic DNA can be effected, e.g., by using the well-known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide.

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including variant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus those phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and variant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMR1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminurn, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenaturn, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianurn, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a polypeptide of the present invention, comprising (a) cultivating a cell, which in its wild-type form produces the polypeptide, under conditions conducive for production of the polypeptide; and optionally, (b) recovering the polypeptide.

The present invention also relates to methods of producing a polypeptide of the present invention, comprising (a) cultivating a recombinant host cell of the present invention under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides having hydrolytic activity. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### Fermentation Broth Formulations or Cell Compositions

The present invention also relates to a fermentation broth formulation or a cell composition comprising a polypeptide of the present invention. The fermentation broth product further comprises additional ingredients used in the fermentation process, such as, for example, cells (including, the host cells containing the gene encoding the polypeptide of the present invention which are used to produce the polypeptide of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In some embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

### Enzyme Compositions

The invention relates to compositions comprising a polypeptide of the present invention in combination with one or more additional component(s). In a preferred embodiment the polypeptides to be used in the compositions comprise one or more or all of the motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO:40). The choice of additional components is within the skill of the artisan and includes conventional ingredients, including the exemplary non-limiting components set forth below.

Some embodiment of the invention relates to a composition comprising:
a) at least 0.001 ppm of at least one polypeptide having hydrolytic activity, wherein the polypeptide is selected for the group consisting of: SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36 and polypeptides having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity hereto;
b) one or more adjunct ingredient.

Some embodiment of the invention relates to a cleaning composition comprising:
a) at least 0.001 ppm of at least one polypeptide having hydrolytic activity, wherein the polypeptide is selected for the group consisting of: SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33 and polypeptides having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity hereto;
b) one or more cleaning composition component, preferably selected from surfactants, builders, bleach components, polymers, dispersing agents and additional enzymes.

The choice of cleaning components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

Surfactants

The detergent composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and may include any conventional surfactant(s) known in the art.

When included therein the detergent will usually contain from about 1% to about 40% by weight of an anionic surfactant, such as from about 5% to about 30%, including from about 5% to about 15%, or from about 15% to about 20%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.

When included therein the detergent will usually contain from about 1% to about 40% by weigh of a cationic surfactant, for example from about 0.5% to about 30%, in particular from about 1% to about 20%, from about 3% to about 10%, such as from about 3% to about 5%, from about 8% to about 12% or from about 10% to about 12%. Non-limiting examples of cationic surfactants include alkyldimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, ester quats, and combinations thereof.

When included therein the detergent will usually contain from about 0.2% to about 40% by weight of a nonionic surfactant, for example from about 0.5% to about 30%, in particular from about 1% to about 20%, from about 3% to about 10%, such as from about 3% to about 5%, from about 8% to about 12%, or from about 10% to about 12%. Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

When included therein the detergent will usually contain from about 0.1% to about 10% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, and combinations thereof.

When included therein the detergent will usually contain from about 0.1% to about 10% by weight of a zwitterionic surfactant. Non-limiting examples of zwitterionic surfactants include betaines such as alkyldimethylbetaines, sulfobetaines, and combinations thereof.

Builders and Co-Builders

The detergent composition may contain about 0-65% by weight, such as about 5% to about 50% of a detergent builder or co-builder, or a mixture thereof. In a dish wash detergent, the level of builder is typically 40-65%, particularly 50-65%. The builder and/or co-builder may particularly be a chelating agent that forms water-soluble complexes with Ca and Mg. Any builder and/or co-builder known in the art for use in cleaning detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as 2,2′-iminodiethan-1-ol), triethanolamine (TEA, also known as 2,2′,2″-nitrilotriethan-1-ol), and (carboxymethyl)inulin (CMI), and combinations thereof.

The detergent composition may also contain 0-50% by weight, such as about 5% to about 30%, of a detergent co-builder. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA), N-(2-hydroxyethyl)ethylenediamine-N,N′,N″-triacetic acid (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053.

Bleaching Systems

The detergent may contain 0-30% by weight, such as about 1% to about 20%, of a bleaching system. Any bleaching system comprising components known in the art for use in cleaning detergents may be utilized. Suitable bleaching system components include sources of hydrogen peroxide; sources of peracids; and bleach catalysts or boosters.

Sources of Hydrogen Peroxide:

Suitable sources of hydrogen peroxide are inorganic persalts, including alkali metal salts such as sodium percarbonate and sodium perborates (usually mono- or tetrahydrate), and hydrogen peroxide-urea (1/1).

Sources of Peracids:

Peracids may be (a) incorporated directly as preformed peracids or (b) formed in situ in the wash liquor from hydrogen peroxide and a bleach activator (perhydrolysis) or (c) formed in situ in the wash liquor from hydrogen peroxide and a perhydrolase and a suitable substrate for the latter, e.g., an ester.

a) Suitable preformed peracids include, but are not limited to, peroxycarboxylic acids such as peroxybenzoic acid and its ring-substituted derivatives, peroxy-α-naphthoic acid, peroxyphthalic acid, peroxylauric acid, peroxystearic acid, ε-phthalimidoperoxycaproic acid [phthalimidoperoxyhexanoic acid (PAP)], and o-carboxybenzamidoperoxycaproic acid; aliphatic and aromatic diperoxydicarboxylic acids such as diperoxydodecanedioic acid, diperoxyazelaic acid, diperoxysebacic acid, diperoxybrassylic acid, 2-decyldiperoxybutanedioic acid, and diperoxyphthalic, -isophthalic and -terephthalic acids; perimidic acids; peroxymonosulfuric acid; peroxydisulfuric acid; peroxyphosphoric acid; peroxysilicic acid; and mixtures of said compounds. It is understood that the peracids mentioned may in some cases be best added as suitable salts, such as alkali metal salts (e.g., Oxone®) or alkaline earth-metal salts.

b) Suitable bleach activators include those belonging to the class of esters, amides, imides, nitriles or anhydrides and, where applicable, salts thereof. Suitable examples are tetraacetylethylenediamine (TAED), sodium 4-[(3,5,5-trimethylhexanoyl)oxy]benzene-1-sulfonate (ISONOBS), sodium 4-(dodecanoyloxy)benzene-1-sulfonate (LOBS), sodium 4-(decanoyloxy)benzene-1-sulfonate, 4-(decanoyloxy)benzoic acid (DOBA), sodium 4-(nonanoyloxy)benzene-1-sulfonate (NOBS), and/or those disclosed in WO98/17767. A particular family of bleach activators of interest was disclosed in EP624154 and particularly preferred in that family is acetyl triethyl citrate (ATC). ATC or a short chain triglyceride like triacetin has the advantage that they are environmentally friendly. Furthermore, acetyl triethyl citrate and triacetin have good hydrolytical stability in the product upon storage and are efficient bleach activators. Finally, ATC is multifunctional, as the citrate released in the perhydrolysis reaction may function as a builder.

Bleach Catalysts and Boosters

The bleaching system may also include a bleach catalyst or booster.
Some non-limiting examples of bleach catalysts that may be used in the compositions of the present invention include manganese oxalate, manganese acetate, manganese-collagen, cobalt-amine catalysts and manganese triazacyclononane (MnTACN) catalysts; particularly preferred are complexes of manganese with 1,4,7-trimethyl-1,4,7-triazacyclononane (Me3-TACN) or 1,2,4,7-tetramethyl-1,4,7-triazacyclononane (Me4-TACN), in particular Me3-TACN, such as the dinuclear manganese complex [(Me3-TACN)Mn(O)3Mn(Me3-TACN)](PF6)2, and [2,2′,2″-nitrilotris(ethane-1,2-diylazanylylidene-κN-methanylylidene)triphenolato-κ3O]manganese(III). The bleach catalysts may also be other metal compounds; such as iron or cobalt complexes.

In some embodiments, where a source of a peracid is included, an organic bleach catalyst or bleach booster may be used having one of the following formulae:

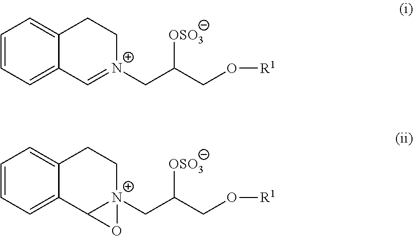

(iii) and mixtures thereof; wherein each R1 is independently a branched alkyl group containing from 9 to 24 carbons or linear alkyl group containing from 11 to 24 carbons, preferably each R1 is independently a branched alkyl group containing from 9 to 18 carbons or linear alkyl group containing from 11 to 18 carbons, more preferably each R1 is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl.

Other exemplary bleaching systems are described, e.g. in WO2007/087258, WO2007/087244, WO2007/087259, EP1867708 (Vitamin K) and WO2007/087242. Suitable photobleaches may for example be sulfonated zinc or aluminium phthalocyanines.

Metal Care Agents

Metal care agents may prevent or reduce the tarnishing, corrosion or oxidation of metals, including aluminium, stainless steel and non-ferrous metals, such as silver and copper. Suitable examples include one or more of the following:

(a) benzatriazoles, including benzotriazole or bis-benzotriazole and substituted derivatives thereof. Benzotriazole derivatives are those compounds in which the available substitution sites on the aromatic ring are partially or completely substituted. Suitable substituents include linear or branch-chain Ci-C20-alkyl groups (e.g., C1-C20-alkyl groups) and hydroxyl, thio, phenyl or halogen such as fluorine, chlorine, bromine and iodine.

(b) metal salts and complexes chosen from the group consisting of zinc, manganese, titanium, zirconium, hafnium, vanadium, cobalt, gallium and cerium salts and/or complexes, the metals being in one of the oxidation states II, III, IV, V or VI. In one aspect, suitable metal salts and/or metal complexes may be chosen from the group consisting of Mn(II) sulphate, Mn(II) citrate, Mn(II) stearate, Mn(II) acetylacetonate, K\hat{ }TiF6 (e.g., K2TiF6), K\hat{ }ZrF6 (e.g., K2ZrF6), CoSO4, Co(NOs)2 and Ce(NOs)3, zinc salts, for example zinc sulphate, hydrozincite or zinc acetate.;

(c) silicates, including sodium or potassium silicate, sodium disilicate, sodium metasilicate, crystalline phyllosilicate and mixtures thereof.

Further suitable organic and inorganic redox-active substances that act as silver/copper corrosion inhibitors are disclosed in WO 94/26860 and WO 94/26859. Preferably the composition of the invention comprises from 0.1 to 5% by weight of the composition of a metal care agent, preferably the metal care agent is a zinc salt.

Hydrotropes

The detergent may contain 0-10% by weight, for example 0-5% by weight, such as about 0.5 to about 5%, or about 3% to about 5%, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Suitable examples include PVP-K15, PVP-K30, ChromaBond S-400, ChromaBond S-403E and Chromabond S-100 from Ashland Aqualon, and Sokalan® HP 165, Sokalan® HP 50 (Dispersing agent), Sokalan® HP 53 (Dispersing agent), Sokalan® HP 59 (Dispersing agent), Sokalan® HP 56 (dye transfer inhibitor), Sokalan® HP 66 K (dye transfer inhibitor) from BASF. Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated. Particularly preferred polymer is ethoxylated homopolymer Sokalan® HP 20 from BASF, which helps to prevent redeposition of soil in the wash liquor.

Fabric Hueing Agents

The detergent compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g. WO 2007/087257 and WO2007/087243.

Enzymes

The detergent additive as well as the detergent composition may comprise one or more additional enzymes such as one or more lipase, cutinase, an amylase, carbohydrase, cellulase, pectinase, mannanase, arabinase, galactanase, xylanase, oxidase, e.g., a laccase, and/or peroxidase.

In general, the properties of the selected enzyme(s) should be compatible with the selected detergent, (i.e., pH-optimum, compatibility with other enzymatic and non-enzymatic ingredients, etc.), and the enzyme(s) should be present in effective amounts.

Cellulases

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. Nos. 4,435,307, 5,648,263, 5,691,178, 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. Nos. 5,457,046, 5,686,593, 5,763,254, WO 95/24471, WO 98/12307 and WO99/001544.

Other cellulases are endo-beta-1,4-glucanase enzyme having a sequence of at least 97% identity to the amino acid sequence of position 1 to position 773 of SEQ ID NO:2 of WO 2002/099091 or a family 44 xyloglucanase, which a xyloglucanase enzyme having a sequence of at least 60% identity to positions 40-559 of SEQ ID NO: 2 of WO 2001/062903.

Commercially available cellulases include Celluzyme™, and Carezyme™ (Novozymes NS) Carezyme Premium™ (Novozymes NS), Celluclean™ (Novozymes NS), Celluclean Classic™ (Novozymes NS), Cellusoft™ (Novozymes NS), Whitezyme™ (Novozymes NS), Clazinase™, and Puradax HA™ (Genencor International Inc.), and K\hat{ }C-500(B)™ (Kao Corporation).

Mannanases

Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 1999/064619. A commercially available mannanase is Mannaway (Novozymes NS).

Peroxidases/Oxidases

Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257. Commercially available peroxidases include Guardzyme™ (Novozymes NS).

Lipases and Cutinases:

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include Lipolase™, Lipex™; Lipolex™ and Lipoclean™ (Novozymes NS), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

Amylases:

Suitable amylases include alpha-amylases and/or a glucoamylases and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48A+T49I+G107A+H156Y+A181T+N190F+I201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, I206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T1651, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T131I+T1651+K178L+T182G+Y305R+G475K wherein the variants are C-terminally truncated and optionally further comprises a substitution at position 243 and/or a deletion at position 180 and/or position 181.

Further suitable amylases are amylases having SEQ ID NO: 1 of WO13184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, 1203, S241, R458, T459, D460, G476 and G477. More preferred variants of SEQ ID NO: 1 are those having the substitution in one of more of the following positions: K176L, E187P, N192FYH, M199L, 1203YF, S241QADN, R458N, T459S, D460T, G476K and G477K and/or deletion in position R178 and/or S179 or of T180 and/or G181. Most preferred amylase variants of SEQ ID NO: 1 are those having the substitutions:

E187P+1203Y+G476K

E187P+1203Y+R458N+T459S+D460T+G476K

wherein the variants optionally further comprise a substitution at position 241 and/or a deletion at position 178 and/or position 179.

Further suitable amylases are amylases having SEQ ID NO: 1 of WO10104675 or variants having 90% sequence identity to SEQ ID NO: 1 thereof. Preferred variants of SEQ ID NO: 1 are those having a substitution, a deletion or an insertion in one of more of the following positions: N21, D97, V128 K177, R179, S180, 1181, G182, M200, L204, E242, G477 and G478. More preferred variants of SEQ ID NO: 1 are those having the substitution in one of more of the following positions: N21D, D97N, V128I K177L, M200L, L204YF, E242QA, G477K and G478K and/or deletion in position R179 and/or S180 or of 1181 and/or G182. Most preferred amylase variants of SEQ ID NO: 1 are those having the substitutions:

N21D+D97N+V128I

wherein the variants optionally further comprise a substitution at position 200 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™, Stainzyme Plus™, Natalase™, Liquozyme X and BAN™ (from Novozymes NS), and Rapidase™, Purastar™/Effectenz™, Powerase, Preferenz S1000, Preferenz S100 and Preferenz S110 (from Genencor International Inc./DuPont).

### Proteases:

Suitable proteases include those of bacterial, fungal, plant, viral or animal origin e.g. vegetable or microbial origin. Microbial origin is preferred. Chemically modified or protein engineered mutants are included. It may be an alkaline protease, such as a serine protease or a metalloprotease. A serine protease may for example be of the S1 family, such as trypsin, or the S8 family such as subtilisin. A metalloproteases protease may for example be a thermolysin from e.g. family M4 or other metalloprotease such as those from M5, M7 or M8 families.

The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family.

Examples of subtilases are those derived from Bacillus such as Bacillus lentus, B. alkalophilus, B. subtilis, B. amyloliquefaciens, Bacillus pumilus and Bacillus gibsonii described in; U.S. Pat. No. 7,262,042 and WO09/021867, and subtilisin lentus, subtilisin Novo, subtilisin Carlsberg, Bacillus licheniformis, subtilisin BPN′, subtilisin 309, subtilisin 147 and subtilisin 168 described in WO89/06279 and protease PD138 described in (WO93/18140). Other useful proteases may be those described in WO 92/175177, WO 01/016285, WO 02/026024 and WO 02/016547. Examples of trypsin-like proteases are trypsin (e.g. of porcine or bovine origin) and the Fusarium protease described in WO 89/06270, WO 94/25583 and WO 05/040372, and the chymotrypsin proteases derived from Cellumonas described in WO 05/052161 and WO 05/052146.

A further preferred protease is the alkaline protease from Bacillus lentus DSM 5483, as described for example in WO 95/23221, and variants thereof which are described in WO 92/21760, WO 95/23221, EP 1921147 and EP 1921148.

Examples of metalloproteases are the neutral metalloprotease as described in WO07/044993 (Genencor Int.) such as those derived from Bacillus amyloliquefaciens.

Examples of useful proteases are the variants described in: WO 92/19729, WO 96/034946, WO 98/20115, WO 98/20116, WO 99/011768, WO 01/44452, WO 03/006602, WO 04/03186, WO 04/041979, WO 07/006305, WO 11/036263, WO 11/036264, especially the variants with substitutions in one or more of the following positions: 3, 4, 9, 15, 24, 27, 42, 55, 59, 60, 66, 74, 85, 96, 97, 98, 99, 100, 101, 102, 104, 116, 118, 121, 126, 127, 128, 154, 156, 157, 158, 161, 164, 176, 179, 182, 185, 188, 189, 193, 198, 199, 200, 203, 206, 211, 212, 216, 218, 226, 229, 230, 239, 246, 255, 256, 268 and 269 wherein the positions correspond to the positions of the Bacillus lentus protease shown in SEQ ID NO 1 of WO 2016/001449. More preferred the subtilase variants may comprise one or more of the mutations: S3T, V41, S9R, S9E, A15T, S24G, S24R, K27R, N42R, S55P, G59E, G59D, N60D, N60E, V66A, N74D, N85S, N85R, G96S, G96A, S97G, S97D, S97A, S97SD, S99E, S99D, S99G, S99M, S99N, S99R, S99H, S101A, V1021, V102Y, V102N, S104A, G116V, G116R, H118D, H118N, N120S, S126L, P127Q, S128A, S154D, A156E, G157D, G157P, S158E, Y161A, R164S, Q176E, N179E, S182E, Q185N, A188P, G189E, V193M, N198D, V1991, Y203W, 5206G, L211Q, L211D, N212D, N2125, M2165, A226V, K229L, Q230H, Q239R, N246K, N255W, N255D, N255E, L256E, L256D T268A or R269H. The protease variants are preferably variants of the Bacillus lentus protease (Savinase®) shown in SEQ ID NO 1 of WO 2016/001449, the Bacillus amyloliquefaciens protease (BPN′) shown in SEQ ID NO 2 of WO2016/001449. The protease variants preferably have at least 80% sequence identity to SEQ ID NO 1 or SEQ ID NO 2 of WO 2016/001449.

A protease variant comprising a substitution at one or more positions corresponding to positions 171, 173, 175, 179, or 180 of SEQ ID NO: 1 of WO2004/067737, wherein said protease variant has a sequence identity of at least 75% but less than 100% to SEQ ID NO: 1 of WO 2004/067737.

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Blaze®, Blaze Evity® 100T, Blaze Evity® 125T, Blaze Evity® 150T, Neutrase®, Everlase® and Esperase® (Novozymes NS), those sold under the tradename Maxatase®, Maxacal®, Maxapem®, Purafect Ox®, Purafect OxP®, Puramax®, FN2®, FN3®, FN4®, Excellase®, Excellenz P1000™, Excellenz P1250™, Eraser®, Preferenz P100™, Purafect Prime®, Preferenz P110™, Effectenz P1000™, Purafect®™, Effectenz P1050™, Purafect Ox®™, Effectenz P2000™, Purafast®, Properase®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and K\hat{ }P (Bacillus alkalophilus subtilisin) from Kao.

### Peroxidases/Oxidases

A peroxidase according to the invention is a peroxidase enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257.

A suitable peroxidase includes a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions. Preferably, the haloperoxidase is a vanadium haloperoxidase, i.e., a vanadate-containing haloperoxidase. Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis. Haloperoxidases have also been isolated from bacteria such as Pseudomonas, e.g., P. pyrrocinia and Streptomyces, e.g., S. aureofaciens.

A suitable oxidase includes in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5). Preferred laccase enzymes are enzymes of microbial origin. The enzymes may be derived from plants, bacteria or fungi (including filamentous fungi and yeasts). Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885). Suitable examples from bacteria include a laccase derivable from a strain of Bacillus. A laccase derived from Coprinopsis or Myceliophthora is preferred; in particular, a laccase derived from Coprinopsis cinerea, as disclosed in WO 97/08325; or from Myceliophthora thermophila, as disclosed in WO 95/33836.

Dispersants

The detergent compositions of the present invention can also contain dispersants. In particular, powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

Dye Transfer Inhibiting Agents

The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Fluorescent Whitening Agent

The detergent compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2.2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diary) pyrazolines and the 7-alkylaminocoumarins. Suitable fluorescent brightener levels include lower levels of from about 0.01, from 0.05, from about 0.1 or even from about 0.2 wt % to upper levels of 0.5 or even 0.75 wt %.

Soil Release Polymers

The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers is amphiphilic alkoxylated grease cleaning polymers comprising a core structure and a plurality of alkoxylate groups attached to that core structure. The core structure may comprise a polyalkylenimine structure or a polyalkanolamine structure as described in detail in WO2009/087523 (hereby incorporated by reference). Furthermore, random graft co-polymers are suitable soil release polymers. Suitable graft co-polymers are described in more detail in WO2007/138054, WO2006/108856 and WO2006/113314 (hereby incorporated by reference). Suitable polyethylene glycol polymers include random graft co-polymers comprising: (i) hydrophilic backbone comprising polyethylene glycol; and (ii) side chain(s) selected from the group consisting of: C4-C25 alkyl group, polypropylene, polybutylene, vinyl ester of a saturated C1-C6 mono-carboxylic acid, CI-C 6 alkyl ester of acrylic or methacrylic acid, and mixtures thereof. Suitable polyethylene glycol polymers have a polyethylene glycol backbone with random grafted polyvinyl acetate side chains. The average molecular weight of the polyethylene glycol backbone can be in the range of from 2,000 Da to 20,000 Da, or from 4,000 Da to 8,000 Da. The molecular weight ratio of the polyethylene glycol backbone to the polyvinyl acetate side chains can be in the range of from 1:1 to 1:5, or from 1:1.2 to 1:2. The average number of graft sites per ethylene oxide units can be less than 1, or less than 0.8, the average number of graft sites per ethylene oxide units can be in the range of from 0.5 to 0.9, or the average number of graft sites per ethylene oxide units can be in the range of from 0.1 to 0.5, or from 0.2 to 0.4. A suitable polyethylene glycol polymer is Sokalan HP22. Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

Anti-Redeposition Agents

The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

Rheology Modifiers

The detergent compositions of the present invention may also include one or more rheology modifiers, structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of a liquid detergent composition. The rheology and viscosity of the detergent can be modified and adjusted by methods known in the art, for example as shown in EP 2169040. Other suitable cleaning composition components include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

Formulation of Detergent Products

The detergent composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

Pouches can be configured as single or multicompartments. It can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids: US2009/0011970 A1.

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent. A liquid or gel detergent may be non-aqueous.

### Granular Detergent Formulations

The composition(s) of the invention may be formulated as a granule for example as a co-granule that combines one or more enzymes. Each enzyme will then be present in more granules securing a more uniform distribution of enzymes in the detergent. This also reduces the physical segregation of different enzymes due to different particle sizes. Methods for producing multi-enzyme co-granulates for the detergent industry are disclosed in the IP.com disclosure IPCOM000200739D.

Another example of formulation of enzymes by the use of co-granulates are disclosed in WO 2013/188331, which relates to a detergent composition comprising (a) a multi-enzyme co-granule; (b) less than 10 wt zeolite (anhydrous basis); and (c) less than 10 wt phosphate salt (anhydrous basis), wherein said enzyme co-granule comprises from 10 to 98 wt % moisture sink component and the composition additionally comprises from 20 to 80 wt % detergent moisture sink component. A multi-enzyme co-granule may comprise an Polypeptide of the invention and (a) one or more enzymes selected from lipases, cellulases, xyloglucanases, perhydrolases, peroxidases, lipoxygenases, laccases, hemicellulases, proteases, care cellulases, cellobiose dehydrogenases, xylanases, phospho lipases, esterases, cutinases, pectinases, mannanases, pectate lyases, keratinases, reductases, oxidases, phenoloxidases, ligninases, pullulanases, tannases, pentosanases, lichenases glucanases, arabinosidases, hyaluronidase, chondroitinase, amylases, and mixtures thereof.

In one aspect, the present invention provides a granule, which comprises:
(a) a core comprising a polypeptide comprising the amino acid sequence shown in SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36 or polypeptides having, at least 60%, at least 65%, at least 70%, at least 75%, at least 80%, at least 85%, at least 90%, at least 95%, at least 97%, at least 98%, at least 99% or 100% sequence identity hereto, and
(b) optionally a coating consisting of one or more layer(s) surrounding the core.

### Medical Cleaning

The present invention further relates to methods of cleaning a medical device and to the use of a composition comprising a GH39 glycosyl hydrolases and at least one adjunct ingredient for cleaning of a medical device. The invention further relates to a method of preventing biofilm formation on a medical device e.g. an indwelling medical device or implant comprising coating the device with at least one GH39 glycosyl hydrolase.

One embodiment of the invention relates to a method of preventing biofilm formation on a medical device e.g. an indwelling medical device or implant comprising coating the device with at least one GH39 glycosyl hydrolase.

The polypeptides suitable for use in medical cleaning and in compositions for medical cleaning are described above and include polypeptides which comprises one or more motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO:40) and/or polypeptide selected from the group consisting of polypeptides having the amino acid sequence of SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36 and polypeptides having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity hereto.

One aspect of the invention relates to a method of cleaning a medical device, wherein the method comprises
a) contacting the medical device with the composition comprising a GH39 glycosyl hydrolase, for a period effective to clean the medical device;
b) cleaning, the medical device; and
c) optionally disinfect the medical device.
   One aspect of the invention relates to a method of cleaning a medical device, wherein the method comprises
a) contacting the medical device with the composition comprising a GH39 glycosyl hydrolase, which comprises one or more motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO:40) and/or is selected from the group consisting of GH39 glycosyl hydrolases having the amino acid sequence of SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36 and polypeptides having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity hereto, for a period effective to clean the medical device;
b) cleaning, the medical device; and
c) optionally disinfect the medical device.
   One embodiment relates to a composition comprising a GH39 glycosyl hydrolase, which comprises one or more motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO:40) and/or is selected from the group consisting of GH39 glycosyl hydrolases having the amino acid sequence of SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36 and polypeptides having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity hereto and preferably an adjunct ingredient. The composition may be an anti-biofouling composition and the composition may be a cleaning or pharmaceutical composition. The adjunct ingredient may be any excipient suitable for e.g. cleaning or pharmaceutical compositions. The adjuncts/excipients are within the choice of the skilled artisan. The adjunct ingredient may be selected from the group consisting of surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxido reductases, bluing agents and fluorescent dyes, antioxidants, and solubilizers. The compositions may be used for detaching biofilm or preventing biofilm formation on surfaces such as medical devices. The medical device may be characterized in that at least a portion of a patient-contactable surface of said device is coated with composition comprising a GH39 glycosyl hydrolase of the invention. The medical device or implant may be any device or implant that is susceptible to biofilm formation. The medical device may be selected from the group consisting of a catheter such as a central venous catheter, intravascular catheter, urinary catheter, Hickman catheter, peritoneal dialysis catheter, endrotracheal catheter, or wherein the device is a mechanical heart valve, a cardiac pacemaker, an arteriovenous shunt, a scleral buckle, a prosthetic joint, a tympanostomy tube, a tracheostomy tube, a voice prosthetic, a penile prosthetic, an artificial urinary sphincter, a synthetic pubovaginal sling, a surgical suture, a bone anchor, a bone screw, an intraocular lens, a contact lens, an intrauterine device, an aortofemoral graft, a vascular graft, a needle, a Luer-Lok connector, a needleless connector and a surgical instrument.

### Uses

The polypeptides of the invention having hydrolytic activity may be used for deep cleaning of an item, such as a textile. In a preferred embodiment the polypeptides of the invention comprise one or more of the motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO:40). In some embodiment of the invention relates to the use of a polypeptide according to the invention for prevention reduction or removal of malodor. Some embodiment of the invention relates to the use of a polypeptide of the invention for prevention or reduction of anti-redeposition and improvement of whiteness of a textile subjected to multiple washes. One embodiment of the invention relates to the use of a polypeptide according to the invention for deep cleaning of an item, wherein item is a textile. One embodiment of the invention relates to the use of a polypeptide according to the invention

(i) for preventing, reducing or removing stickiness of the item;

(ii) for pretreating stains on the item;

(iii) for preventing, reducing or removing redeposition of soil during a wash cycle;

(iv) for preventing, reducing or removing adherence of soil to the item;

(v) for maintaining or improving whiteness of the item;

(vi) for preventing, reducing or removal malodor from the item,
wherein the item is a textile.
   One embodiment of the invention relates to the use of a polypeptide according to the invention for deep cleaning of an item, wherein item is a textile. One embodiment of the invention relates to the use of a polypeptide,
(i) for preventing, reducing or removing stickiness of the item;
(ii) for pretreating stains on the item;
(iii) for preventing, reducing or removing redeposition of soil during a wash cycle;
(iv) for preventing, reducing or removing adherence of soil to the item;
(v) for maintaining or improving whiteness of the item;
(vi) for preventing, reducing or removal malodor from the item, optionally wherein the item is a textile, wherein the polypeptide is selected from the group consisting of:
   (a) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 3;
   (b) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 6;
   (c) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 9;
   (d) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 12;
   (e) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 15;
   (f) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 18;
   (g) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 21;
   (h) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 24;
   (i) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 27;
   (j) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 30;
   (k) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 33; and
   (l) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 36.

The invention is further summarized in the following paragraphs:
1. Use of a GH39 polypeptide e.g. a polypeptide comprising a GH39 domain, for cleaning e.g. deep cleaning of an item, wherein the item is a textile.
2. Use according to paragraph 1 for preventing, reducing or removing stickiness of the item.
3. Use according to any of paragraphs 1 or 2 for pre-treating stains on the item.
4. Use according to any of paragraphs 1-3 for preventing, reducing or removing re-deposition of soil during a wash cycle.
5. Use according to any of paragraphs 1-4 for preventing, reducing or removing adherence of soil to the item.
6. Use according to any of the preceding paragraphs for maintaining or improving the whiteness of the item.
7. Use according to any of the preceding paragraphs, wherein a malodor is reduced or removed from the item.
8. Use according to any of the preceding composition paragraphs, wherein the surface is a textile surface.
9. Use according to any of the preceding composition paragraphs, wherein the textile is made of cotton, Cotton/Polyester, Polyester, Polyamide, Polyacryl and/or silk.
10. Use according to any of the preceding paragraphs, wherein the polypeptide comprises one or more of the motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO:40) and/or is a polypeptide of any of paragraphs 48 to 68.
11. A composition comprising a polypeptide comprising one or more of the motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO:40) or a polypeptide of and an adjunct ingredient.
12. Composition according to paragraph 11, wherein the polypeptide is the polypeptide of paragraphs of any of paragraphs 48 to 68.
13. Composition according to any of the preceding composition paragraphs, wherein the detergent adjunct ingredient is selected from the group consisting of surfactants, builders, flocculating aid, chelating agents, dye transfer inhibitors, enzymes, enzyme stabilizers, enzyme inhibitors, catalytic materials, bleach activators, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, builders and co-builders, fabric huing agents, anti-foaming agents, dispersants, processing aids, and/or pigments.
14. Composition according to any of the preceding composition paragraphs wherein the composition comprises from about 5 wt % to about 50 wt %, from about 5 wt % to about 40 wt %, from about 5 wt % to about 30 wt %, from about 5 wt % to about 20 wt %, from about 5 wt % to about 10 wt % anionic surfactant, preferably selected from linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.
15. Composition according to any of the preceding composition paragraphs wherein the composition comprises from about 10 wt % to about 50 wt % of at least one builder, preferably selected from citric acid, methylglycine-N,N-diacetic acid (MGDA) and/or glutamic acid-N,N-diacetic acid (GLDA) and mixtures thereof.
16. Composition according to any of the proceeding paragraphs comprising from about 5 wt % to about 40 wt % nonionic surfactant, and from about 0 wt % to about 5 wt % anionic surfactant.
17. Composition according to paragraph 16, wherein the nonionic surfactant is selected from alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA) and combinations thereof.
18. Composition according to any of the preceding composition paragraphs, wherein the composition further comprises one or more enzymes selected from the group consisting of proteases, lipases, cutinases, amylases, carbohydrases, cellulases, pectinases, mannanases, arabinases, galactanases, xylanases and oxidases.
19. Composition according to any of the preceding composition paragraphs, wherein the composition is a bar, a homogenous tablet, a tablet having two or more layers, a pouch having one or more compartments, a regular or compact powder, a granule, a paste, a gel, or a regular, compact or concentrated liquid.
20. Composition according to any of the preceding composition paragraphs, wherein the composition is a cleaning composition selected from liquid detergent, powder detergent and granule detergent compositions.
21. Composition according to any of the preceding composition paragraphs wherein the polypeptide comprises one or more motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO:40) and wherein the polypeptide is selected from the group consisting of polypeptides having the amino acid sequence of SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36 and polypeptides having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity hereto.
22. Composition according to any of the preceding composition paragraphs wherein the polypeptide comprising one or more motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO:40) and comprises the amino acid sequence shown SEQ ID NO 3 or polypeptides having at least 60%, at least 65%, at least 70%, at least 75%, at least 80%, at least 85%, at least 90%, at least 95%, at least 97%, at least 98%, at least 99% or 100% sequence identity hereto.
23. Composition according to any of the preceding composition paragraphs wherein the polypeptide comprising one or more motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO:40) and comprises the amino acid sequence shown SEQ ID NO 6 or polypeptides having at least 60%, at least 65%, at least 70%, at least 75%, at least 80%, at least 85%, at least 90%, at least 95%, at least 97%, at least 98%, at least 99% or 100% sequence identity hereto.
24. Composition according to any of the preceding composition paragraphs wherein the polypeptide comprising one or more motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO:40) and comprises the amino acid sequence shown SEQ ID NO 9 or polypeptides having at least 60%, at least 65%, at least 70%, at least 75%, at least 80%, at least 85%, at least 90%, at least 95%, at least 97%, at least 98%, at least 99% or 100% sequence identity hereto.
25. Composition according to any of the preceding composition paragraphs wherein the polypeptide comprising one or more motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO:40) and comprises the amino acid sequence shown SEQ ID NO 12 or polypeptides having at least 60%, at least 65%, at least 70%, at least 75%, at least 80%, at least 85%, at least 90%, at least 95%, at least 97%, at least 98%, at least 99% or 100% sequence identity hereto.
26. Composition according to any of the preceding composition paragraphs wherein the polypeptide comprising one or more motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO:40) and comprises the amino acid sequence shown SEQ ID NO 15 or polypeptides having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity hereto.
27. Composition according to any of the preceding composition paragraphs wherein the polypeptide comprising one or more motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO:40) and comprises the amino acid sequence shown SEQ ID NO 18 or polypeptides having at least 60%, at least 65%, at least 70%, at least 75%, at least 80%, at least 85%, at least 90%, at least 95%, at least 97%, at least 98%, at least 99% or 100% sequence identity hereto.
28. Composition according to any of the preceding composition paragraphs wherein the polypeptide comprising one or more motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO:40) and comprises the amino acid sequence shown SEQ ID NO 21 or polypeptides having at least 60%, at least 65%, at least 70%, at least 75%, at least 80%, at least 85%, at least 90%, at least 95%, at least 97%, at least 98%, at least 99% or 100% sequence identity hereto.
29. Composition according to any of the preceding composition paragraphs wherein the polypeptide comprising one or more motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO:40) and comprises the amino acid sequence shown SEQ ID NO 24 or polypeptides having at least 60%, at least 65%, at least 70%, at least 75%, at least 80%, at least 85%, at least 90%, at least 95%, at least 97%, at least 98%, at least 99% or 100% sequence identity hereto.
30. Composition according to any of the preceding composition paragraphs wherein the polypeptide comprising one or more motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO:40) and comprises the amino acid sequence shown SEQ ID NO 27 or polypeptides having at least 60%, at least 65%, at least 70%, at least 75%, at least 80%, at least 85%, at least 90%, at least 95%, at least 97%, at least 98%, at least 99% or 100% sequence identity hereto.
31. Composition according to any of the preceding composition paragraphs wherein the polypeptide comprising one or more motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO:40) and comprises the amino acid sequence shown SEQ ID NO 30 or polypeptides having at least 60%, at least 65%, at least 70%, at least 75%, at least 80%, at least 85%, at least 90%, at least 95%, at least 97%, at least 98%, at least 99% or 100% sequence identity hereto.
32. Composition according to any of the preceding composition paragraphs wherein the polypeptide comprising one or more motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO:40) and comprises the amino acid sequence shown SEQ ID NO 33 or polypeptides having at least 60%, at least 65%, at least 70%, at least 75%, at least 80%, at least 85%, at least 90%, at least 95%, at least 97%, at least 98%, at least 99% or 100% sequence identity hereto.
33. Composition according to any of the preceding composition paragraphs wherein the polypeptide comprising one or more motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO:40) and comprises the amino acid sequence shown SEQ ID NO 36 or polypeptides having at least 60%, at least 65%, at least 70%, at least 75%, at least 80%, at least 85%, at least 90%, at least 95%, at least 97%, at least 98%, at least 99% or 100% sequence identity hereto.
34. A laundering method for laundering an item comprising the steps of:
   a. Exposing an item to a wash liquor comprising a polypeptide of paragraphs 48-68 or a composition according to any of paragraphs 11-33;
   b. Completing at least one wash cycle; and
   c. Optionally rinsing the item,
wherein the item is a textile.
35. A method of treating an item, wherein the item is preferably a textile, said method comprising the steps of:
   a. Exposing an item to a polypeptide selected from the group consisting of a polypeptide having at least 60%, at least 65%, at least 70%, at least 75%, at least 80%, at least 85%, at least 90%, at least 95%, at least 97%, at least 98%, at least 99% or 100% sequence identity to the mature polypeptide of SEQ ID NO: 2, SEQ ID NO:5, SEQ ID NO: 8, SEQ ID NO: 11, SEQ ID NO: 14, SEQ ID NO: 17, SEQ ID NO: 20, SEQ ID NO: 23, SEQ ID NO: 26, SEQ ID NO: 29, SEQ ID NO: 32 or SEQ ID NO 35; a wash liquor comprising said polypeptide or a detergent composition according to any proceeding paragraphs.
36. Method according to any proceeding paragraphs, wherein the pH of the wash liquor is in the range of 1 to 11.
37. Method according to any of the preceding method paragraphs, wherein the pH of the wash liquor is in the range 5.5 to 11, such as in the range of 7 to 9, in the range of 7 to 8 or in the range of 7 to 8.5.
38. Method according to any of the preceding method paragraphs, wherein the temperature of the wash liquor is in the range of 5° C. to 95° C., or in the range of 10° C. to 80° C., in the range of 10° C. to 70° C., in the range of 10° C. to 60° C., in the range of 10° C. to 50° C., in the range of 15° C. to 40° C., in the range of 20° C. to 40° C., in the range of 15° C. to 30° C. or in the range of 20° C. to 30° C.
39. Method according to any of the preceding method paragraphs, wherein the temperature of the wash liquor is from about 20° C. to about 40° C.
40. Method according to any of the preceding method paragraphs, wherein the temperature of the wash liquor is from about 15° C. to about 30° C.
41. Method according to any of the preceding method paragraphs, wherein stains present on the item is pre-treated with a polypeptide of paragraphs 48-68 or a detergent composition according to any of paragraphs 11-33.
42. Method according to any of the preceding method paragraphs, wherein stickiness of the item is reduced.
43. Method according to any of the preceding method paragraphs, wherein redeposition of soil is reduced.
44. Method according to any of the preceding method paragraphs, wherein adherence of soil to the item is reduced or removed.
45. Method according to any of the preceding method paragraphs, wherein whiteness of the item is maintained or improved.
46. Method according to any of the preceding method paragraphs, wherein malodor is reduced or removed from the item.
47. Method according to any of the preceding method paragraphs, wherein the concentration of the polypeptide having hydrolytic activity in the wash liquor is at least 0,001 mg of polypeptide, such as at least 5 mg of protein, preferably at least 10 mg of protein, more preferably at least 15 mg of protein, per liter of wash liquor, optionally the concentration of polypeptide in the wash liquor is in the range 0,002 mg/L to 2 mg/L, such as 0.02 mg/L to 2 mg/L, such as 0.2 mg/L to 2 mg/L or in the range of 0,0001 mg/L to 10 mg/L or in the range of in the range of 0,001 mg/L to 10 mg/L, or in the range of 0.01 mg/L to 10 mg/L, or in in the range of 0.1 mg/L to 10 mg/L per liter of wash liquor, optionally the concentration of the polypeptide of the invention is 0.0001% to 2 wt %, such as 0.001 to 0.1 wt %, such as 0.005 to 0.1 wt %, such as 0.01 to 0.1 wt %, such as 0.01 to 0.5 wt % or most preferred 0.002 to 0.09 wt % in the total detergent concentration.
48. A polypeptide having hydrolytic activity, selected from the group consisting of:
   a. a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 2, SEQ ID NO:5, SEQ ID NO: 8, SEQ ID NO: 11, SEQ ID NO: 14, SEQ ID NO: 17, SEQ ID NO: 20, SEQ ID NO: 23, SEQ ID NO: 26, SEQ ID NO: 29, SEQ ID NO: 32, SEQ ID NO 35 or a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide shown in SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36;
   b. a polypeptide encoded by a polynucleotide that hybridizes under low stringency conditions with
      i. the mature polypeptide coding sequence of SEQ ID NO 1, SEQ ID NO 4, SEQ ID NO 7, SEQ ID NO 10, SEQ ID NO 13, SEQ ID NO 16, SEQ ID NO 19, SEQ ID NO 22, SEQ ID NO 25, SEQ ID NO 28, SEQ ID NO 31, SEQ ID NO 34;
      ii. the cDNA sequence thereof, or
      iii. the full-length complement of (i) or (ii);
   c. a polypeptide encoded by a polynucleotide having at least 60%, at least 65%, at least 70%, at least 75%, at least 80%, at least 85%, at least 90%, at least 95%, at least 97%, at least 98%, at least 99% or 100% sequence identity to the mature polypeptide coding sequence of SEQ ID NO 1, SEQ ID NO 4, SEQ ID NO 7, SEQ ID NO 10, SEQ ID NO 13, SEQ ID NO 16, SEQ ID NO 19, SEQ ID NO 22, SEQ ID NO 25, SEQ ID NO 28, SEQ ID NO 31, SEQ ID NO 35 or the cDNA sequence thereof;
   d. a variant of the mature polypeptide shown in SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36 comprising a substitution, deletion, and/or insertion at one or more positions or a variant of the mature polypeptide shown in SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36 comprising a substitution, deletion, and/or insertion at one or more positions;
   e. a fragment of the polypeptide of (a), (b), (c), or (d) and which have hydrolytic activity and;
   f. a polypeptide comprising one or more or all of the motif(s) [A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO:40).
49. The polypeptide of paragraph 48, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 2, SEQ ID NO:5, SEQ ID NO: 8, SEQ ID NO: 11, SEQ ID NO: 14, SEQ ID NO: 17, SEQ ID NO: 20, SEQ ID NO: 23, SEQ ID NO: 26, SEQ ID NO: 29, SEQ ID NO: 32 or SEQ ID NO 35 or to the mature polypeptide shown in SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36.
50. The polypeptide of paragraph 48 or 49, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 2 or to the mature polypeptide shown in SEQ ID NO: 3.
51. The polypeptide of paragraph 48 or 49, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 5 or to the mature polypeptide shown in SEQ ID NO: 6.
52. The polypeptide of paragraph 48 or 49, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 8 or to the mature polypeptide shown in SEQ ID NO: 9.
53. The polypeptide of paragraph 48 or 49, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 11 or to the mature polypeptide shown in SEQ ID NO: 12.
54. The polypeptide of paragraph 48 or 49, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 14 or to the mature polypeptide shown in SEQ ID NO: 15.
55. The polypeptide of paragraph 48 or 49, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 17 or to the mature polypeptide shown in SEQ ID NO: 18.
56. The polypeptide of paragraph 48 or 49, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 20 or to the mature polypeptide shown in SEQ ID NO: 21.
57. The polypeptide of paragraph 48 or 49, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 23 or to the mature polypeptide shown in SEQ ID NO: 24.
58. The polypeptide of paragraph 48 or 49, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 26 or to the mature polypeptide shown in SEQ ID NO: 27.
59. The polypeptide of paragraph 48 or 49, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 29 or to the mature polypeptide shown in SEQ ID NO: 30.
60. The polypeptide of paragraph 48 or 49, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 32 or to the mature polypeptide shown in SEQ ID NO: 33.
61. The polypeptide of paragraph 48 or 49, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 32 or to the mature polypeptide shown in SEQ ID NO: 36.
62. The polypeptide according to any of paragraphs 48 to 61, which is encoded by a polynucleotide that hybridizes under low stringency conditions, low-medium stringency conditions, medium stringency conditions, medium-high stringency conditions, high stringency conditions, or very high stringency conditions with
   i. the mature polypeptide coding sequence of SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28 or SEQ ID NO: 31, SEQ ID NO 34;
   ii. the cDNA sequence thereof, or
   iii. the full-length complement of (i) or (ii).
63. The polypeptide according to any of paragraphs 48 to 62, which is encoded by a polynucleotide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide coding sequence of SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31 or SEQ ID NO 34 or the cDNA sequence thereof.
64. The polypeptide according to any of paragraphs 48 to 63, comprising or consisting of SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36 or the mature polypeptide of SEQ ID NO 2, SEQ ID NO 5, SEQ ID NO 8, SEQ ID NO 11, SEQ ID NO 14, SEQ ID NO 17, SEQ ID NO 20, SEQ ID NO 23, SEQ ID NO 26, SEQ ID NO 29, SEQ ID NO 32, SEQ ID NO 35.
65. The polypeptide according to any of paragraphs 48 to 64, which is a variant of the any of the polypeptides with SEQ ID NO 3, SEQ ID NO 6, SEQ ID NO 9, SEQ ID NO 12, SEQ ID NO 15, SEQ ID NO 18, SEQ ID NO 21, SEQ ID NO 24, SEQ ID NO 27, SEQ ID NO 30, SEQ ID NO 33, SEQ ID NO 36 comprising a substitution, deletion, and/or insertion at one or more positions.
66. The polypeptide according to any of preceding paragraphs for use as a medicament.
67. The polypeptide according to any of proceeding paragraphs for use in treatment or prevention of a bacterial infection, preferably said bacterial infection is an infection caused by Gram-positive or Gram-negative bacteria, further preferably said bacterial infection is selected from a group consisting of: Staphylococcus spp. (e.g., Staphylococcus epidermidis, S. aureus), Enterococcus spp. (e.g., Enterococcus faecalis), Escherichia spp. (e.g., Escherichia cob), Listeria spp. (e.g., Listeria monocytogenes), Pseudomonas spp. (e.g., Pseudomonas aeruginosa), Bacillus spp., Salmonella spp., Coagulase-negative Staphylococci, Klebsiella spp. (e.g., Klebsiella pneumoniae) infections.
68. The polypeptide according to any of proceeding paragraphs for use in treatment or prevention of a disease selected from the group consisting of: Cystic fibrosis pneumonia (e.g., caused by Pseudomonas aeruginosa and/or Burkholderia cepacia), Meloidosis (e.g., caused by Pseudomonas pseudomallei), Necrotizing fasciitis (e.g., caused by Group A streptococci), Musculoskeletal infections (e.g., caused by Staphylococci and other Gram-positive cocci), Otitis media (e.g., caused by Haemophilus influenzae), Biliary tract infection (e.g., caused by E. coli and other enteric bacteria), Urinary catheter cystitis (e.g., caused by E. coli and other Gram-negative rods), Bacterial prostatitis (e.g., E. coli and other Gram-negative bacteria), Periodontitis (e.g., caused by Gram negative anaerobic oral bacteria), Dental caries (e.g., caused by Streptococcus spp. and other acidogenic Gram positive cocci).
69. A polynucleotide encoding the polypeptide according to any of paragraphs 48-68.
70. A nucleic acid construct or expression vector comprising the polynucleotide of paragraph 69 operably linked to one or more control sequences that direct the production of the polypeptide in an expression host.
71. A recombinant host cell comprising the polynucleotide of paragraph 69 operably linked to one or more control sequences that direct the production of the polypeptide.
72. A method of producing the polypeptide of any of paragraphs 48-68, comprising cultivating a cell, which in its wild-type form produces the polypeptide, under conditions conducive for production of the polypeptide.
73. The method of paragraph 72, further comprising recovering the polypeptide.
74. A method of producing a polypeptide according to any of paragraphs 48-68, comprising cultivating the host cell of paragraph 71 under conditions conducive for production of the polypeptide.
75. The method of paragraph 74, further comprising recovering the polypeptide.
76. A nucleic acid construct or expression vector comprising a gene encoding a protein operably linked to the polynucleotide of paragraph 69, wherein the gene is foreign to the polynucleotide encoding the signal peptide.
77. A recombinant host cell comprising a gene encoding a protein operably linked to the polynucleotide of paragraph 69, wherein the gene is foreign to the polynucleotide encoding the signal peptide.
78. A method of producing a protein, comprising cultivating a recombinant host cell comprising a gene encoding a protein operably linked to the polynucleotide of paragraph 69, wherein the gene is foreign to the polynucleotide encoding the signal peptide, under conditions conducive for production of the protein.
79. The method of paragraph 78, further comprising recovering the protein.
80. Item laundered according to the method of any of paragraphs 34-47.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### EXAMPLES

### Model Detergents

Model detergent A wash liquor (100%) was prepared by dissolving 3.33 g/l of model detergent A containing 12% LAS, 1.1% AEO Biosoft N25-7 (NI), 7% AEOS (SLES), 6% MPG, 3% ethanol, 3% TEA (triethanolamine), 2.75% cocoa soap, 2.75% soya soap, 2% glycerol, 2% sodium hydroxide, 2% sodium citrate, 1% sodium formiate, 0.2% DTMPA and 0.2% PCA (all percentages are w/w (weight volume) in water with hardness 15 dH.

Triple-20 Nonionic Model Detergent (60% surfactant) was prepared by dissolving 3.33 g/I non-ionic detergent containing NaOH 0.87%, MPG (Monopropylenglycol) 6%, Glycerol 2%, Soap-soy 2.75%, Soap-coco 2.75%, PCA (Sokalon CP-5) 0.2%, AEO Biosoft N25-7(NI) 16%, Sodium formiate 1%, Sodium Citrate 2%, DTMPA 0.2%, Ethanol (96%) 3%, adjustment of pH with NaOH or Citric acid as water to 100% (all percentages are w/w (weight volume) in water with hardness 15 dH.

Model Detergent MC: A medical cleaning model detergent (model detergent MC) was prepared containing 5% MPG (propylene glycol), 5% Pluronic PE 4300 (PO/EO block polymer; 70%/30%, approx. 1750 g/mol), 2% Plurafac LF 305 (fatty alcohol alkoxylate; C6-10+EO/PO), 1% MGDA (methyl glycine diacetic acid, 1% TEA (triethanolamine) (all percentages are w/w). The pH was adjusted to 8.7 with phosphoric acid.

### Wash Assays

### Mini Launder-O-Meter (MiniLOM) Model Wash System

MiniLOM is a modified mini wash system of the Launder-O-Meter (LOM), which is a medium scale model wash system that can be applied to test up to 20 different wash conditions simultaneously. A LOM is basically a large temperature controlled water bath with 20 closed metal beakers rotating inside it. Each beaker constitutes one small washing machine and during an experiment, each will contain a solution of a specific detergent/enzyme system to be tested along with the soiled and unsoiled fabrics it is tested on. Mechanical stress is achieved by the beakers being rotated in the water bath and by including metal balls in the beaker.

The LOM model wash system is mainly used in medium scale testing of detergents and enzymes at European wash conditions. In a LOM experiment, factors such as the ballast to soil ratio and the fabric to wash liquor ratio can be varied. Therefore, the LOM provides the link between small scale experiments, such as AMSA and mini-wash, and the more time consuming full scale experiments in front loader washing machines.

In miniLOM, washes are performed in 50 ml test tubes placed in Stuart rotator.

### Example 1 Cloning and Expression of Polypeptides of the Invention

The DNA encoding the gene of SEQ ID NO 1, SEQ ID NO 4, SEQ ID NO 7, SEQ ID NO 10, SEQ ID NO 13, SEQ ID NO 16, SEQ ID NO 19, SEQ ID NO 22, SEQ ID NO 25, SEQ ID NO 28, SEQ ID NO 31, SEQ ID NO 34 were isolated from bacterial strains isolated from soil samples collected in different countries (see table 1). Chromosomal DNA from the different strains was subjected to full genome sequencing using Illumine technology. The genome sequence was analyzed for protein sequences that that had glycosyl hydrolase domains (according to the CAZY definition). 11 GH39 glycosyl hydrolase genes and corresponding sequence were identified in the genomes.

TABLE 1

| Mature | | country |
|---|---|---|
| protein | donor | of origin |
| SEQ ID NO 3 | Pseudomonas fluorescens | Iceland |
| SEQ ID NO 6 | Pseudomonas sp-62165 | Denmark |
| SEQ ID NO 9 | Luteolibacter sp-62326 | Denmark |
| SEQ ID NO 12 | Pseudomonas sp-62430 | United States |
| SEQ ID NO 15 | Pseudomonas frederiksbergensis | Sweden |
| SEQ ID NO 18 | Rhodococcus globerulus | Denmark |
| SEQ ID NO 21 | Paenibacillus daejeonensis | Malaysia |
| SEQ ID NO 24 | Pseudomonas sp-62168 | Denmark |
| SEQ ID NO 27 | Dyella sp-62115 | Denmark |
| SEQ ID NO 30 | Pseudomonas fulva | Sweden |
| SEQ ID NO 33 | Rahnella sp-62576 | Sweden |
| SEQ ID NO 36 | Pseudomonas aeruginosa | Australia |

### Example 2: Cloning and Expression of Polypeptides of the Invention

The DNA encoding the mature peptide of GH39 genes SEQ ID NO 1, SEQ ID NO 4, SEQ ID NO 7, SEQ ID NO 10, SEQ ID NO 13, SEQ ID NO 16, SEQ ID NO 19, SEQ ID NO 22, SEQ ID NO 25, SEQ ID NO 28, SEQ ID NO 31, SEQ ID NO 34 were amplified from the genomic DNA of the corresponding bacterial strains by standard PCR techniques using specific primers containing an overhang to cloning vector. The amplified PCR fragments were inserted into a Bacillus expression vector as described in WO 12/025577. Briefly, the DNA encoding the mature peptide of the gene was cloned in frame to a Bacillus clausii secretion signal (BcSP; with the following amino acid sequence: MKKPLGKIVASTALLISVAFSSSIASA (SEQ ID NO: 41 (former SEQ ID NO 38)). BcSP replaced the native secretion signal in the gene. Downstream of the BcSP sequence, an affinity tag sequence was introduced to ease the purification process (His-tag; with the following amino acid sequence: HHHHHHPR (SEQ ID NO: 42 (former SEQ ID NO 39)) The gene that was expressed therefore comprised the BcSP sequence followed by the His-tag sequence followed by the mature wild type GH39 gene sequence. The final expression plasmid (BcSP-His-tag-GH39) was transformed into a Bacillus subtilis expression host. The GH39 BcSP-fusion gene was integrated by homologous recombination into the Bacillus subtilis host cell genome upon transformation. The gene construct was expressed under the control of a triple promoter system (as described in WO 99/43835). The gene coding for chloramphenicol acetyltransferase was used as maker (as described in (Diderichsen et al., 1993, Plasmid 30: 312-315)). Transformants were selected on LB media agar supplemented with 6 microgram of chloramphenicol per ml. One recombinant Bacillus subtilis clone containing the GH39 expression construct was selected and was cultivated on a rotary shaking table in 500 ml baffled Erlenmeyer flasks each containing 100 ml yeast extract-based media. After 3-5 days cultivation time at 30° C. to 37° C., the enzyme containing supernatant was harvested by centrifugation and the enzymes was purified by His-tag purification.

### Example 3: His Tag Purification Method

The His-tagged GH39 enzymes were purified by immobilized metal chromatography (IMAC) using Ni²⁺ as the metal ion on 5 mL HisTrap Excel columns (GE Healthcare Life Sciences). The purification took place at pH 7 and the bound protein was eluted with imidazole. The purity of the purified enzymes was checked by SDS-PAGE and the concentration of the enzyme determined by Absorbance 280 nm after a buffer exchange in 50 mM HEPES, 100 mM NaCl pH7.0

### Example 4: MiniLom Deep-Cleaning in Liquid Model Detergent on Psi Swatches

A crude extract of the biofilm extracellular polymer Psi was prepared from Pseudomonas aeruginosa (DSM 22644) as follows; The strain was restreaked on LB agar and incubated for 3 days at 37° C. A single colony was then used to inoculated 100 ml of Tryptic Soy Broth (aliquoted into tubes containing 10 ml each), and the tubes were incubated overnight at 37° C. The cultures were then pooled, and pelleted by centrifugation (10 min, 6000 g, 25° C.). The pellet was resuspended in 3 M sodium chloride, vortexed vigorously and incubated for 15 min at ambient temperature to extract the surface-associated polymer. The cells were then re-pelleted (10 min, 16000 g, 25° C.) and the Psl-containing supernatant was retrieved. This crude extract was stored at −20° C. until further use (termed Psi extract).

Wash performance was determined as follows; 50 ul aliquots of the crude Psi extract were spotted on sterile textile swatches (WFK20A, 65% polyester/35% cotton) and incubated for 15 min at ambient temperature. The swatches (with sodium chloride or extract) were placed in 50 mL test tubes and 10 mL of wash liquor (15° dH water with 0.2 g/L iron(III) oxide nano-powder (544884; Sigma-Aldrich) with 3.33 g/L liquid model detergent) and the enzyme(s) (when appropriate) was added to each tube. Washes without enzyme were included as controls. The test tubes were placed in a Stuart rotator and incubated for 1 hour at 30° C. and 20 rpm. The wash liquor was then removed, and the swatches were rinsed twice with 15° dH water and dried on filter paper over night.

The color difference (L) values were measured using a Handheld Minolta CR-300, and are displayed in table 2. Delta values (L_{(swatch washed with enzyme)}−L_{(swatch washed without enzyme)}) are also indicated.

TABLE 2
Deep-cleaning effects of the PslG homologues
in non-ionic model detergent.

| | | | | ΔL (L^{with enzyme} − |
|---|---|---|---|---|
| | | | L values, | ^{Lwithout enzyme}), |
| | | Enzyme | non-ionic | non-ionic |
| | | concentration | model | model |
| Substrate | Enzyme | (ppm) | detergent | detergent |
| Wfk20A | | 0 | 88.8 | |
| swatch, | | | | |
| 3M salt | | | | |
| Psl extract, no | | 0 | 81.5 | |
| enzyme | | | | |
| Psl extract | SEQ ID | 10.0 | 84.4 | 2.9 |
| | NO 21 | | | |
| Psl extract | SEQ ID | 10.0 | 83.4 | 1.9 |
| | NO 15 | | | |
| Psl extract | SEQ ID | 10.0 | 86.1 | 4.6 |
| | NO 9 | | | |
| Psl extract | SEQ ID | 10.0 | 88.6 | 7.1 |
| | NO 6 | | | |
| Psl extract | SEQ ID | 10.0 | 84.9 | 3.4 |
| | NO 12 | | | |
| Psl extract | SEQ ID | 5.0 | 85.3 | 3.8 |
| | NO 18 | | | |
| Psl extract | SEQ ID | 10.0 | 85.3 | 3.8 |
| | NO 24 | | | |
| Psl extract | SEQ ID | 10.0 | 84.3 | 2.8 |
| | NO 27 | | | |
| Psl extract | SEQ ID | 10.0 | 87.8 | 6.3 |
| | NO 33 | | | |
| Psl extract | SEQ ID | 10.0 | 82.1 | 0.6 |
| | NO 30 | | | |

### Example 5: Construction of Clades and Phylogenetic Trees

The GH39 domain includes the polypeptides of the invention having activity on Psi and comprises the GH39 domain as well as the clusters such as the clades. A phylogenetic tree was constructed, of polypeptide sequences containing a GH39 domain, as defined in the CAZY database (Lombard, Henrissat et al, 2014. The carbohydrate-active enzymes database (CAZy) in 2013. Nucleic Acids Res. 42, http://www.cazy.org/). The phylogenetic tree was constructed from a multiple alignment of mature polypeptide sequences containing at least one GH39 domain. The sequences were aligned using the MUSCLE algorithm version 3.8.31 (Edgar, 2004. Nucleic Acids Research 32(5): 1792-1797), and the trees were constructed using FastTree version 2.1.8 (Price et al., 2010, PloS one 5(3)) and visualized using iTOL (Letunic & Bork, 2007. Bioinformatics 23(1): 127-128). The polypeptide comprises of the GH39 domain comprises several motifs one example is [A/G/S]XHPY (SEQ ID NO 37) situated in positions corresponding to positions 205 to 209 in Pseudomonas fluorescens (SEQ ID NO 3). The H at the position corresponding to position 207 of SEQ ID NO 3 and Y at position 209 are predicted to be involved in substrate binding. Another motif which may be comprised by the polypeptides of the invention is [I/V/L/F/M][Y/W/F]X[T/S]EXG (SEQ ID NO: 38), corresponding to positions 242 to 248 in SEQ ID NO 3.

The polypeptides containing a GH39 domain can be separated into distinct sub-clusters. The sub-clusters are defined by one or more short sequence motifs, as well as containing a GH39 domain as defined in the CAZY database (Lombard, Henrissat et al, 2014). We denoted one sub-cluster comprising the motif [A/G/S]XHPY (SEQ ID NO 37) as the HPY clade. All polypeptide sequences containing a GH39 domain as well as the motif will be denoted as belonging to the HPY clade.

The polypeptides in the HPY clade can be further separated into multiple distinct sub-clusters, or clades, where we denoted the clades listed below. The distinct motifs for each clade are described in detail below.

### Generation of HPY Domain

A phylogenetic tree was constructed, of polypeptide sequences containing a GH39 domain, as defined above. The phylogenetic tree was constructed from a multiple alignment of mature polypeptide sequences containing at least one GH39 domain. The sequences were aligned using the MUSCLE algorithm version 3.8.31 (Edgar, 2004. Nucleic Acids Research 32(5): 1792-1797), and the tree was constructed using FastTree version 2.1.8 (Price et al., 2010, PloS one 5(3)) and visualized using iTOL (Letunic & Bork, 2007. Bioinformatics 23(1): 127-128). The polypeptides in GH39 can be separated into at least distinct sub-clusters, one where denoted HPY. A characteristic motif for this subgroup is the motif [A/G/S]XHPY (SEQ ID NO 37) corresponding to amino acid 205 to 209 in the reference polypeptide (SEQ ID NO 3). The H at the position corresponding to position 207 of SEQ ID NO 3 and Y at position 209 are predicted to be involved in substrate binding. Another motif characteristic of this domain is [I/V/L/F/M][Y/W/F]X[T/S]EXG (SEQ ID NO 38), corresponding to position 242 to 248 in SEQ ID NO 3. An additional motif characteristic of this domain is [D/G/I/V]XXX[E/Q][I/L/V]WNE[P/Q/W/F] (SEQ ID NO 39), corresponding to position 127 to 136 in SEQ ID NO 3, where N and E, at positions 134 and 135 are predicted to be involved in substrate binding.

### Generation of Phylogenetic Trees

A phylogenetic tree was constructed, of polypeptide sequences containing a HPY domain, as defined above. The phylogenetic tree was constructed from a multiple alignment of mature polypeptide sequences containing at least one HPY domain. The sequences were aligned using the MUSCLE algorithm version 3.8.31 (Edgar, 2004. Nucleic Acids Research 32(5): 1792-1797), and the tree was constructed using FastTree version 2.1.8 (Price et al., 2010, PloS one 5(3)) and visualized using iTOL (Letunic & Bork, 2007, Bioinformatics 23(1): 127-128). The polypeptides in HPY can be separated into multiple distinct sub-clusters, or clades, where we denoted the clades listed below. The distinct motifs for each clade are described in details below.

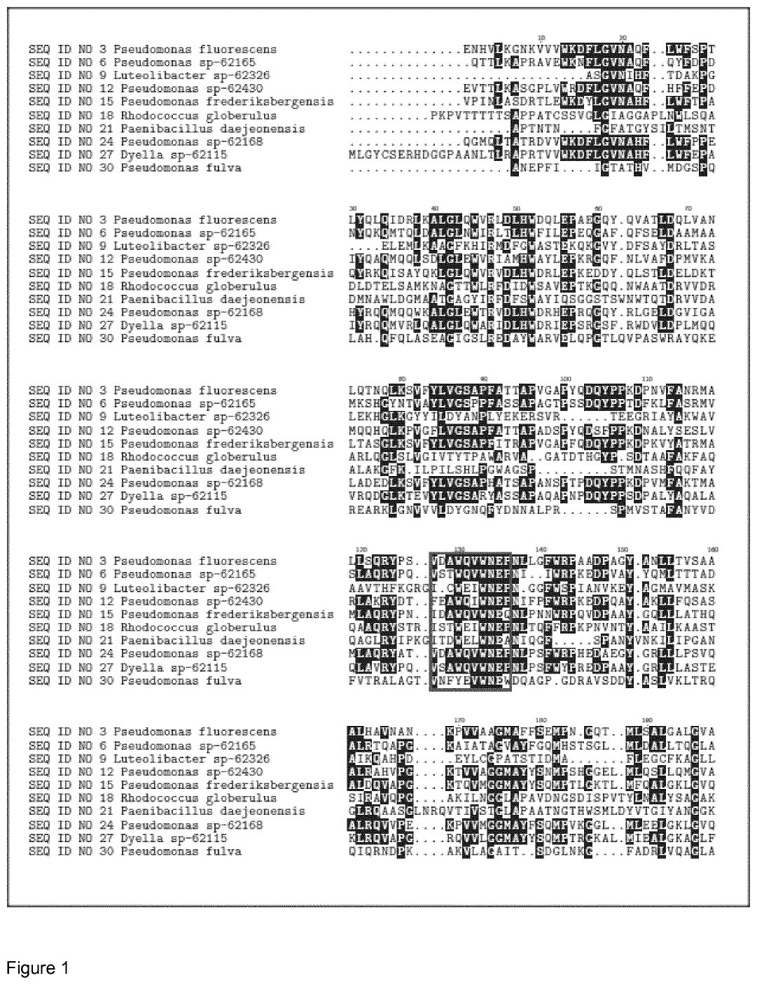

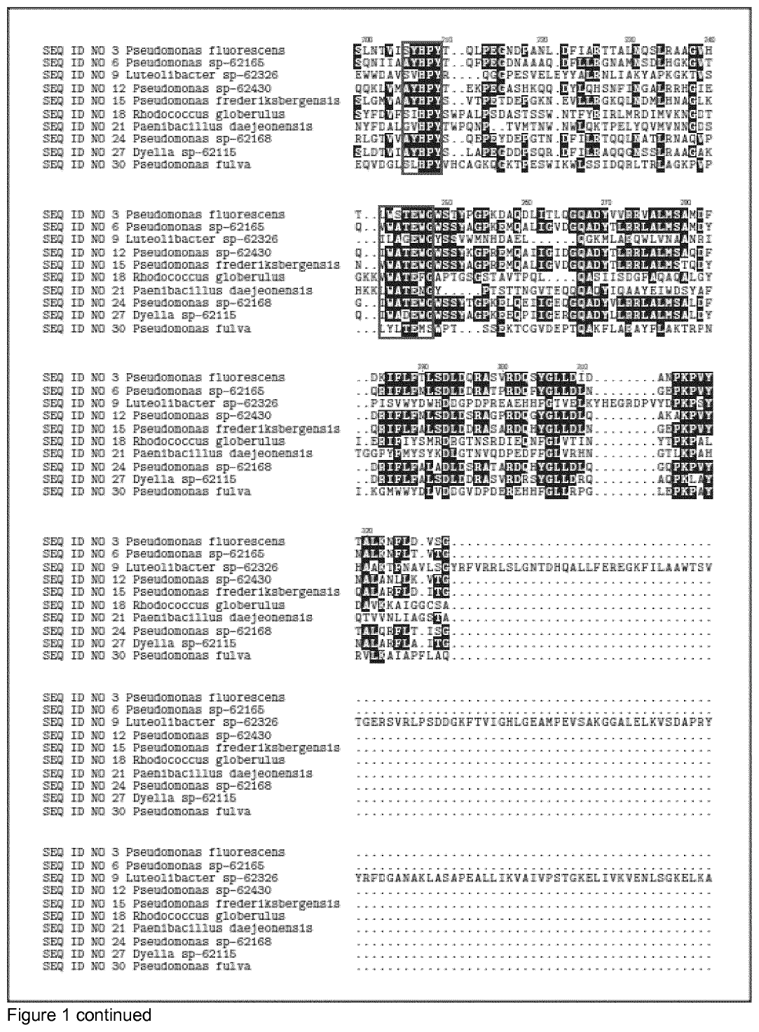

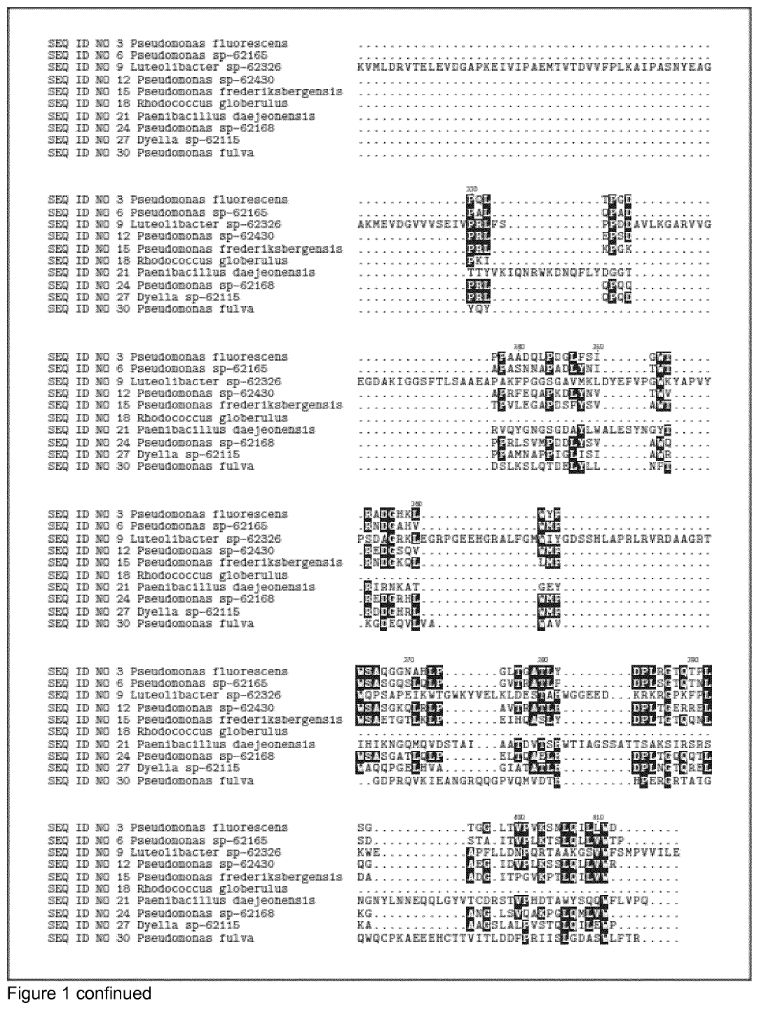

An alignment of the polypeptides of the HPY domain is shown in FIG. 1.

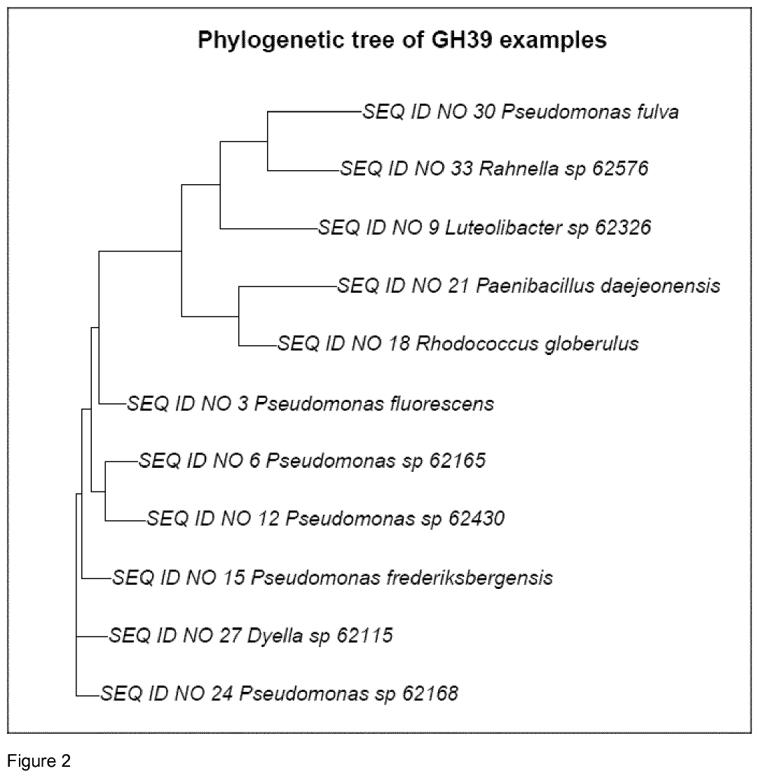

A phylogenetic tree of the polypeptides of the invention is shown in FIG. 2.

### Generation of WQVW Clade

The WQVW clade comprises GH39 polypeptides of bacterial origin, belonging to the HPY clade and having activity on Psl. The polypeptides of the clade comprise the motif example [ANTV]WQVW (SEQ ID NO: 37), corresponding to pos 129 to 133 of Pseudomonas fluorescens (SEQ ID NO 3).

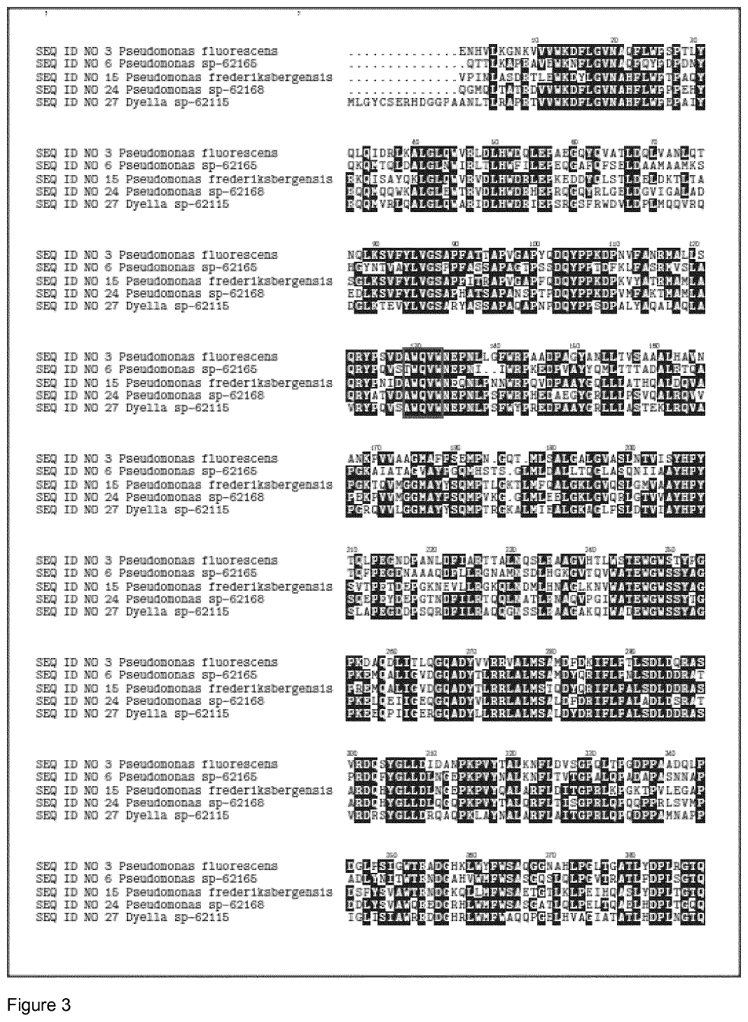

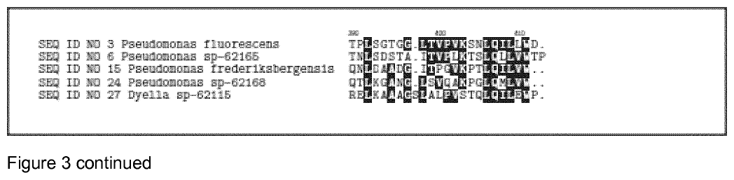

An alignment of the polypeptides of the invention comprised in the clade is shown in FIG. 3.

### Example 6 Biofilm Removal Activity in Medium and in Liquid Model Detergent

Pseudomonas aeruginosa PAO1 strains DSM19880 and DSM22644 were used as model microorganisms in the present example. The strains were restreaked on LB agar and incubated at 30° C. Single colonies were inoculated into 10 mL LBNS (LB no salt) and the cultures were incubated for 16 hours at 37° C. under shaking conditions. The cultures were subsequently diluted (1:100) in LBNS, added to 96-well microtiter plates (150 μL per well, Thermo Scientific, cat #167008) and Peg lids were inserted (NUNC-TSP, Thermo Scientific, cat #445497). The plates were then incubated for 24 hours at 26° C. under static conditions. After incubation, the peg lids were rinsed in MTP plates with 5° dH water hardness, and transferred to treatment plates with medium (LBNS) or model cleaning solution (5 g/L Model detergent MC in 5° dH water hardness) containing no enzyme (control) or 20 μg/mL enzyme for 1 hour at 26° C. The lids were subsequently rinsed in water hardness and stained with 0.095% crystal violet (Sigma-Aldrich, cat # V5265) for 15 min. Following the staining, the peg lids were rinsed twice, moved to clean microtiter plates and the remaining dye was dissolved with 30% acetic acid. The absorbance was measured at 595 nm. The results are displayed in table 2 and 3

TABLE 2
Biofilm removal activity in LBNS medium

| | | | Remaining |
|---|---|---|---|
| | | Enzyme dosage | biofilm (% of |
| Strain | Enzyme | (μg/ml) | untreated control) |
| DSM19880 | No enzyme | 0 | 100.0 |
| DSM19880 | SEQ ID NO 36 | 20 | 2.5 |
| DSM19880 | SEQ ID NO 3 | 20 | 1.6 |
| DSM22644 | No enzyme | 0 | 100.0 |
| DSM22644 | SEQ ID NO 36 | 20 | 11.7 |
| DSM22644 | SEQ ID NO 3 | 20 | 17.1 |

TABLE 3
Biofilm removal in medical cleaning model detergent MC

| | | | Remaining |
|---|---|---|---|
| | | Enzyme dosage | biofilm (% of |
| Strain | Enzyme | (μg/ml) | untreated control) |
| DSM19880 | No enzyme | 0 | 100.0 |
| DSM19880 | SEQ ID NO 36 | 20 | 3.8 |
| DSM19880 | SEQ ID NO 3 | 20 | 5.9 |
| DSM22644 | No enzyme | 0 | 100.0 |
| DSM22644 | SEQ ID NO 36 | 20 | 3.9 |
| DSM22644 | SEQ ID NO 3 | 20 | 5.2 |

### Example 7 Endoscope Cleaning in Liquid Model Detergent

Endoscope biofilms were established using P. aeruginosa DSM19880: The strain was inoculated into 10 mL LBNS (LB no salt) and incubated at 37° C. for 16 hours with shaking (200 rpm). After propagation, the culture was diluted (1:100) in LBNS and the bacterial suspension was added to 96-well microtiter plates (Thermo Scientific, cat #167008) containing sterile pieces (1 cm) of endoscope tubing (4.7 mm diameter, Fluoroelastomer/Viton®, USP Class VI, Endoscopy Development Company, LLC). Sterile medium was added to control wells. After 24h at 26° C. (static incubation), the endoscope tubes were treated with a model cleaning solution (5 g/L Model detergent MC in 5° dH water hardness) containing no enzyme (control) or 20 μg/mL enzyme for 1 hour at 26° C. The endoscope pieces were subsequently rinsed with 5° dH water and stained with 0.095% crystal violet (SIGMA V5265) for 15 min. After additional rinses, the endoscope pieces were blotted on absorbent paper and the remaining dye was dissolved using 30% acetic acid. 200 μl aliquots of the suspensions were transferred to a 96-well microtiter plate and the absorbance was measured at 595 nm. The results are displayed in table 4 as percentages of remaining biofilm after enzymatic treatment as compared to the control (endoscope biofilm treated without enzyme).

TABLE 4
Endoscope cleaning properties in medical
cleaning model detergent MC

| | | Remaining |
|---|---|---|
| | Enzyme dosage | biofilm (% of |
| Enzyme | (μg/ml) | untreated control) |
| No enzyme | 0 | 100.0 |
| SEQ ID NO 36 | 20 | 32.3 |
| SEQ ID NO 3 | 20 | 18.0 |

The results show that the polypeptides tested have endoscope cleaning properties i.e. disrupt and/or remove the biofilm or components of the biofilm tested when compared to samples comprising no enzyme.

1. A GH39 polypeptide having hydrolytic activity, selected from the group consisting of:
(a) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 3;
(b) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 6;
(c) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 9;
(d) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 12;
(e) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 15;
(f) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 18;
(g) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 21;
(h) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 24;
(i) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 27;
(j) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 30;
(k) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 33;
(l) a polypeptide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 36;
(m) a variant of the polypeptide selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO 36 wherein the variant has hydrolytic activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19 or 20 positions;
(n) a polypeptide comprising the polypeptide of (a) to (I) and a N-terminal and/or C-terminal His-tag and/or HQ-tag;
(o) a polypeptide comprising the polypeptide of (a) to (I) and a N-terminal and/or C-terminal extension of between 1 and 10 amino acids;
(p) a fragment of the polypeptide of (a) to (I) having hydrolytic activity and having at least 90% of the length of the mature polypeptide
(q) a polypeptide comprising one or more or all of the motif(s)[A/G/S]XHPY (SEQ ID NO 37) or [I/V/L/F/M][Y/W/F]X[T/S]EXG (SEQ ID NO 38), [D/G/I/V]XXX[E/Q][I/L/V]WNE[P/Q/W/F] (SEQ ID NO 39) or [ANTV]WQVW (SEQ ID NO:40).

2. The polypeptide of claim 1, having at least 65%, sequence identity to the polypeptide shown in SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33 or SEQ ID NO 36.

3. The polypeptide of claim 1, which is encoded by a polynucleotide having at least 65%, sequence identity to the mature polypeptide coding sequence of SEQ ID NO 1, SEQ ID NO: 4, SEQ ID NO: 7, SEQ ID NO: 10, SEQ ID NO: 13, SEQ ID NO 16, SEQ ID NO: 19, SEQ ID NO: 22, SEQ ID NO: 25, SEQ ID NO: 28, SEQ ID NO: 31 or SEQ ID NO 34.

4. The polypeptide of claim 1 selected from the group consisting of polypeptides: (a) comprising or consisting of SEQ ID NO: 3 or the mature polypeptide of SEQ ID NO: 2;
(b) comprising or consisting of SEQ ID NO: 6 or the mature polypeptide of SEQ ID NO: 5;
(c) comprising or consisting of SEQ ID NO: 9 or the mature polypeptide of SEQ ID NO: 8;
(d) comprising or consisting of SEQ ID NO: 12 or the mature polypeptide of SEQ ID NO: 11;
(e) comprising or consisting of SEQ ID NO: 15 or the mature polypeptide of SEQ ID NO: 14.
(f) comprising or consisting of SEQ ID NO: 18 or the mature polypeptide of SEQ ID NO: 17;
(g) comprising or consisting of SEQ ID NO: 21 or the mature polypeptide of SEQ ID NO: 20;
(h) comprising or consisting of SEQ ID NO: 24 or the mature polypeptide of SEQ ID NO: 23;
(i) comprising or consisting of SEQ ID NO: 27 or the mature polypeptide of SEQ ID NO: 26;
(j) comprising or consisting of SEQ ID NO: 30 or the mature polypeptide of SEQ ID NO: 29;
(k) comprising or consisting of SEQ ID NO: 33 or the mature polypeptide of SEQ ID NO: 32;
(l) comprising or consisting of SEQ ID NO: 36 or the mature polypeptide of SEQ ID NO: 35.

5. A polynucleotide encoding the polypeptide of claim 1.

6. A nucleic acid construct or expression vector comprising the polynucleotide of claim 5 operably linked to one or more control sequences that direct the production of the polypeptide in an expression host.

7. A recombinant host cell comprising the polynucleotide of claim 5 operably linked to one or more control sequences that direct the production of the polypeptide.

8. A method of producing the polypeptide of claim 1, comprising cultivating a cell, which in its wild-type form produces the polypeptide, under conditions conducive for production of the polypeptide.

9. The method of claim 8, further comprising recovering the polypeptide.

10. A method of producing a polypeptide having hydrolytic activity, comprising cultivating the host cell of claim 7 under conditions conducive for production of the polypeptide.

11. The method of claim 10, further comprising recovering the polypeptide.

12. A composition comprising the polypeptide of claim 1.

13. The composition according to claim 12, wherein the composition is a cleaning or ADW composition.

14. (canceled)

15. (canceled)

16. A laundering method for laundering an item comprising the steps of:
a. exposing an item to a wash liquor comprising a polypeptide of claim 1;
b. completing at least one wash cycle; and
c. optionally rinsing the item,
wherein the item is a textile.


SEQUENCE LISTING (42 sequences)

SEQ ID NO: 1 - DNA, 1329 bp, Pseudomonas fluorescens
  CDS (1)..(1326)
  sig_peptide (1)..(90)
  mat_peptide (91)..(1326)
atggcagctaaacgcacgtggttagccaccctggccgtggtcgcggcaatcggcctcagcgtctttatgtgggggcgtcaggccgatgccgaaaaccatgtgctcaagggcaacaaggtggtggtgtggaaggactttctgggggtgaatgcgcagttcctgtggttcagcccgacgctgtatcagctgcaaatcgaccgtctcaaggctctgggcctgcaatgggtgcgcctggatctgcattgggaccaactggagcccgctgagggccagtatcaggtcgcgaccctggatcagttggtcgccaacctgcaaaccaaccagctcaaatcggtgttctacctggtgggctcggcgcctttcgccactaccgcgccggtcggtgcgccgtatcaggaccagtacccgcccaaggacccgaatgtgttcgccaaccgcatggcgttgttgtcgcaacgctaccccagcgtcgatgcctggcaagtctggaacgagcccaacctgctcggcttctggcgaccggcggccgacccggccggctatgccaacttgctcaccgtcagcgccgccgctttgcacgcagtgaatgccaacaaaccggtggtggccgccggcatggcgttcttcagcgaaatgcccaacggccagaccatgctctcggcccttggcgcgttgggtgtggccagtttgaacacggtgatttcctatcacccttacacgcagttgcccgaaggcaacgacccggcgaacctggactttatcgccaggaccacggcgctcaaccagtcccttcgcgccgctggcgtgcacaccctgtggagcaccgagtggggttggtcgacctaccccggccccaaagacgcccaagacctgattaccttgcagggccaggccgattatgtagtgcgccgcgtggcgctgatgagcgcgatggatttcgacaagatcttcctgttcaccttgagcgacctcgaccagcgcgccagcgtgcgcgaccagtcttacggcttgctcgacatcgacgccaaccccaagccggtctacaccgccttgaagaactttctcgacgtcagcggcccgcaactcacacccggcgacccgcccgccgccgatcaattgccggacggcttgttcagcatcggctggacccgcgccgacggccacaaactctggtatttctggtcggcccagggcggcaacgcgcacttgcccggtttgaccggcgcgaccctgtacgacccgctgcgcggcacgcaaaccccgctgagtggcaccggcggcctgaccgtaccggtcaagtcgaacctgcaaattctgttatgggattga

SEQ ID NO: 2 - protein, 442 aa, Pseudomonas fluorescens
MAAKRTWLATLAVVAAIGLSVFMWGRQADAENHVLKGNKVVVWKDFLGVNAQFLWFSPTLYQLQIDRLKALGLQWVRLDLHWDQLEPAEGQYQVATLDQLVANLQTNQLKSVFYLVGSAPFATTAPVGAPYQDQYPPKDPNVFANRMALLSQRYPSVDAWQVWNEPNLLGFWRPAADPAGYANLLTVSAAALHAVNANKPVVAAGMAFFSEMPNGQTMLSALGALGVASLNTVISYHPYTQLPEGNDPANLDFIARTTALNQSLRAAGVHTLWSTEWGWSTYPGPKDAQDLITLQGQADYVVRRVALMSAMDFDKIFLFTLSDLDQRASVRDQSYGLLDIDANPKPVYTALKNFLDVSGPQLTPGDPPAADQLPDGLFSIGWTRADGHKLWYFWSAQGGNAHLPGLTGATLYDPLRGTQTPLSGTGGLTVPVKSNLQILLWD

SEQ ID NO: 3 - protein, 412 aa, Pseudomonas fluorescens
ENHVLKGNKVVVWKDFLGVNAQFLWFSPTLYQLQIDRLKALGLQWVRLDLHWDQLEPAEGQYQVATLDQLVANLQTNQLKSVFYLVGSAPFATTAPVGAPYQDQYPPKDPNVFANRMALLSQRYPSVDAWQVWNEPNLLGFWRPAADPAGYANLLTVSAAALHAVNANKPVVAAGMAFFSEMPNGQTMLSALGALGVASLNTVISYHPYTQLPEGNDPANLDFIARTTALNQSLRAAGVHTLWSTEWGWSTYPGPKDAQDLITLQGQADYVVRRVALMSAMDFDKIFLFTLSDLDQRASVRDQSYGLLDIDANPKPVYTALKNFLDVSGPQLTPGDPPAADQLPDGLFSIGWTRADGHKLWYFWSAQGGNAHLPGLTGATLYDPLRGTQTPLSGTGGLTVPVKSNLQILLWD

SEQ ID NO: 4 - DNA, 1326 bp, Pseudomonas sp-62165
  CDS (1)..(1323)
  sig_peptide (1)..(90)
  mat_peptide (91)..(1323)
atgtggcgtaaagtctttttgtggctgccggccctgttgctgatggtggtcgccgtcagcctgatcccctggagccccaacgtcgccgcccagaccacgctcaaggcgccccgcgcagtggagtggaaaaactttctcggggtcaacgcacagttccagtatttcgatccggacaactaccagaagcagatgacgcagctcgacgcgctgggcttgaactggatacgcctgaccctgcattggttcatcctcgaacccgaacagggggctttccagttcagcgaactcgatgctgccatggcggcgatgaaaagccatggctacaacaccgtcgcctacctggtcggctcgccgccgttcgccagcagcgccccggccggcaccccgagcagcgaccagtacccaccgactgacttcaagctgttcgcctcgcgcatggtcagcctggcccagcggtacccacaggtgagcacctggcaggtgtggaacgaaccgaacatcatctggcggcccaaggaagaccccgtggcctactaccagatgctgaccaccaccgccgatgcccttcgcacccaggcgccgggcaaagccatcgctaccgccggcgtcgcttatttcggccagatgcacagcacttccgggctgatgctcgatgccctgctgacccagggcctggccagccagaacatcatcgccgcctatcacccctatacccagtttccggaaggcgacaacgccgcggcccaggacttcctgctcaggggcaacgccatgaacagcgatctgcatggcaagggtgtcacccaggtctgggccaccgaatggggctggtcgagctatgccgggcccaaggaaatgcaggcccttatcggcgtcgacggacaggccgactacaccctgcggcgcctggccctgatgagcgccatggactaccagcggattttcctgttcaacctcagcgacctggatgatcgggccaccccacgcgaccagttttacggcctgctcgacctcaacggcgagccgaagccggtgtataacgccctgaagaacttcctcacggtcaccggcccggccctccagccggccgatgccccggcgtcgaacaacgcacccgccgacctctacaacatcacctggacccgcaacgacggcgcccatgtctggatgttctggagcgccagcggccagagcctgcaactgcccggcgtgacccgggcgacgctgttcgacccgctgagcggtacccagacaaatctgagtgacagcacagccattaccgtgccgctgaaaaccagcctgcagctattggtgtggacgccatga

SEQ ID NO: 5 - protein, 441 aa, Pseudomonas sp-62165
MWRKVFLWLPALLLMVVAVSLIPWSPNVAAQTTLKAPRAVEWKNFLGVNAQFQYFDPDNYQKQMTQLDALGLNWIRLTLHWFILEPEQGAFQFSELDAAMAAMKSHGYNTVAYLVGSPPFASSAPAGTPSSDQYPPTDFKLFASRMVSLAQRYPQVSTWQVWNEPNIIWRPKEDPVAYYQMLTTTADALRTQAPGKAIATAGVAYFGQMHSTSGLMLDALLTQGLASQNIIAAYHPYTQFPEGDNAAAQDFLLRGNAMNSDLHGKGVTQVWATEWGWSSYAGPKEMQALIGVDGQADYTLRRLALMSAMDYQRIFLFNLSDLDDRATPRDQFYGLLDLNGEPKPVYNALKNFLTVTGPALQPADAPASNNAPADLYNITWTRNDGAHVWMFWSASGQSLQLPGVTRATLFDPLSGTQTNLSDSTAITVPLKTSLQLLVWTP

SEQ ID NO: 6 - protein, 411 aa, Pseudomonas sp-62165
QTTLKAPRAVEWKNFLGVNAQFQYFDPDNYQKQMTQLDALGLNWIRLTLHWFILEPEQGAFQFSELDAAMAAMKSHGYNTVAYLVGSPPFASSAPAGTPSSDQYPPTDFKLFASRMVSLAQRYPQVSTWQVWNEPNIIWRPKEDPVAYYQMLTTTADALRTQAPGKAIATAGVAYFGQMHSTSGLMLDALLTQGLASQNIIAAYHPYTQFPEGDNAAAQDFLLRGNAMNSDLHGKGVTQVWATEWGWSSYAGPKEMQALIGVDGQADYTLRRLALMSAMDYQRIFLFNLSDLDDRATPRDQFYGLLDLNGEPKPVYNALKNFLTVTGPALQPADAPASNNAPADLYNITWTRNDGAHVWMFWSASGQSLQLPGVTRATLFDPLSGTQTNLSDSTAITVPLKTSLQLLVWTP

SEQ ID NO: 7 - DNA, 2079 bp, Luteolibacter sp-62326
  CDS (1)..(2076)
  sig_peptide (1)..(87)
  mat_peptide (88)..(2076)
atgatcggcaaagcgattctctccttatggctcgcgtgtttggcgggcctgtccgcggcggaaaagtttccgccggatgtgacggccgcttccggtgtgaacatccacttcaccgatgcgaagcccggtgagctggaaatgctgaaggccgccggcttcaagcacatccggatggatttcggctgggcctccacggagaaacagaagggtgtttacgatttctccgcctatgaccgcctgacggcgtcattggagaagcacggattgaagggatactacatcctggattatgcgaacccgctttatgaaaaggagcgttcggtgcgcaccgaggagggtcggattgcctacgcgaagtgggcggtggcggcggtgacgcatttcaaaggccgcggcatctgttgggagatctggaatgagccgaatggcggattctggtcgccgatcgcgaatgtgaaggaatatgccgggatggcggtgatggcctcgaaggcgatcaagcaggcccatccggatgagtacctttgtggtcccgccacctcgacgatcgacatggcgtttttggagggatgcttcaaagcgggtctgcttgaatggtgggatgcggtgtccgtgcatccctaccgccaaggcggtccggagtcggtggaactcgaatattatgcgctgagaaatctcatcgcgaaatacgctccgaaagggaaaacggtctccatccttgcgggcgaatggggttattcctcggtctggatgaatcacgacgcggagctgcaagggaagatgctcgcacgccaatggctggtgaacgccgcgaaccgtatcccgatttccgtttggtacgattggcatgatgacgggccggatccacgggaggcggagcatcacttcggaacggtagagctgaaatatcatgagggccgagatccggtctatgatccgaaaccttcctatcatgccgcaaagacgttcaatgcggtgttgagcggatatcggttcgtcagacgactatcactggggaataccgatcatcaggcgctgctgtttgagagggaggggaagttcatcctggcggcctggacgagtgtgaccggggagcgttccgttcgcctcccgagtgacgacggcaaattcacggtgatcggtcatttgggcgaggcgatgccagaggtttccgccaagggcggagccttggaactgaaggtgagcgatgcgccgcgttactaccgtttcgatggggcgaatgcgaaactggcgtccgcgcccgaagcgttgttgatcaaggtcgcgatcgtgcccagcaccgggaaggagcttatcgtcaaagtggagaacctttccggcaaagagctgaaggcgaaggtgatgctggatcgggtgacggaactggaggtggatggcgcgccgaaggagatcgtcattcctgcggagatgacggtcacggatgtggttttcccgctgaaggcgatccctgccagcaattacgaagctggcgcgaagatggaagtggatggtgtggtggtttcggagatcgtgccccgtcttttctctccgccggatgatgcggttctgaaaggtgcccgcgtggtaggtgaaggggacgcgaaaatcggcggatcgttcacactttccgccgccgaagctcccgcgaaatttcccggcggatcgggtgcggtgatgaagctggactatgaatttgtgccgggctggaaatacgcgccggtctacccgagtgatgccggacggaaactggaaggtcgccccggcgaggagcacgggcgtgcgttgttcgggatgtggatctacggcgactccagccatctggcgccccggctccgggtgagggatgccgccggccggacgtggcagccatccgctccggagatcaagtggaccggttggaaatacgtggagctgaaactcgacgaaagcaccgcgcactggggtggtgaggaggacaagcggaagcgcggtcccaagttccccctgaaatgggaagctccgttccttctggataacccgcagcgaaccgccgcgaaaggatcggtctggttctcgatgccggtcgtgattctcgagtga

SEQ ID NO: 8 - protein, 692 aa, Luteolibacter sp-62326
MIGKAILSLWLACLAGLSAAEKFPPDVTAASGVNIHFTDAKPGELEMLKAAGFKHIRMDFGWASTEKQKGVYDFSAYDRLTASLEKHGLKGYYILDYANPLYEKERSVRTEEGRIAYAKWAVAAVTHFKGRGICWEIWNEPNGGFWSPIANVKEYAGMAVMASKAIKQAHPDEYLCGPATSTIDMAFLEGCFKAGLLEWWDAVSVHPYRQGGPESVELEYYALRNLIAKYAPKGKTVSILAGEWGYSSVWMNHDAELQGKMLARQWLVNAANRIPISVWYDWHDDGPDPREAEHHFGTVELKYHEGRDPVYDPKPSYHAAKTFNAVLSGYRFVRRLSLGNTDHQALLFEREGKFILAAWTSVTGERSVRLPSDDGKFTVIGHLGEAMPEVSAKGGALELKVSDAPRYYRFDGANAKLASAPEALLIKVAIVPSTGKELIVKVENLSGKELKAKVMLDRVTELEVDGAPKEIVIPAEMTVTDVVFPLKAIPASNYEAGAKMEVDGVVVSEIVPRLFSPPDDAVLKGARVVGEGDAKIGGSFTLSAAEAPAKFPGGSGAVMKLDYEFVPGWKYAPVYPSDAGRKLEGRPGEEHGRALFGMWIYGDSSHLAPRLRVRDAAGRTWQPSAPEIKWTGWKYVELKLDESTAHWGGEEDKRKRGPKFPLKWEAPFLLDNPQRTAAKGSVWFSMPVVILE

SEQ ID NO: 9 - protein, 663 aa, Luteolibacter sp-62326
ASGVNIHFTDAKPGELEMLKAAGFKHIRMDFGWASTEKQKGVYDFSAYDRLTASLEKHGLKGYYILDYANPLYEKERSVRTEEGRIAYAKWAVAAVTHFKGRGICWEIWNEPNGGFWSPIANVKEYAGMAVMASKAIKQAHPDEYLCGPATSTIDMAFLEGCFKAGLLEWWDAVSVHPYRQGGPESVELEYYALRNLIAKYAPKGKTVSILAGEWGYSSVWMNHDAELQGKMLARQWLVNAANRIPISVWYDWHDDGPDPREAEHHFGTVELKYHEGRDPVYDPKPSYHAAKTFNAVLSGYRFVRRLSLGNTDHQALLFEREGKFILAAWTSVTGERSVRLPSDDGKFTVIGHLGEAMPEVSAKGGALELKVSDAPRYYRFDGANAKLASAPEALLIKVAIVPSTGKELIVKVENLSGKELKAKVMLDRVTELEVDGAPKEIVIPAEMTVTDVVFPLKAIPASNYEAGAKMEVDGVVVSEIVPRLFSPPDDAVLKGARVVGEGDAKIGGSFTLSAAEAPAKFPGGSGAVMKLDYEFVPGWKYAPVYPSDAGRKLEGRPGEEHGRALFGMWIYGDSSHLAPRLRVRDAAGRTWQPSAPEIKWTGWKYVELKLDESTAHWGGEEDKRKRGPKFPLKWEAPFLLDNPQRTAAKGSVWFSMPVVILE

SEQ ID NO: 10 - DNA, 1335 bp, Pseudomonas sp-62430
  CDS (1)..(1332)
  sig_peptide (1)..(90)
  mat_peptide (91)..(1332)
atgtggcgtagaaccctgctttttattccgacattccttttgcttggcctcattctgctggtcttgccctggagtcgtcaggctgatgctgaggtaaccactctaaaggcctcggggcctctggtttggcgggactttctcggcgtcaacgcccagtttcatttcttcgagccggatatctatcaggcgcagatgcagcagctctccgacctaggtctggagtgggtaagaattgccatgcactgggcctatctggagcccaagcgcggccagttcaatctggtggccttcgatcccatggtcaaagccatgcagcaacatcagctgaagccggttggtttcttggtgggctctgcacctttcgccacgactgcgccggccgactcgccctatcaggactccttcccgcccaaggataatgctctgtatagcgagagtctggttcgtctggccaagcgctacgatacgttcgaggcgtggcagatctggaacgagccgaatatttttcctttctggcgtcccaaggaagatccgcaagcctacgcaaaactgctatttcagagtgccagcgcattgcgcgcccatgtgccgggcaagactgtcgtggctggcggcatggcttactacagcaatatgccctcccatggcggcgaactcatgctgcaatccctattgcagatgggggtggcccagcagaagctggtgatggcctaccatccctataccgagaagcctgaaggtgcctcgcacaagcaacaggattatctgcagcattccaatttcatcaatggtgcactgcgtaggcatgggatcgagcagatctgggctacggaatggggctggtccagctacaaggggcctagagagatgcaagcaattatcggcatcgatgggcaggcggactataccctgcgcaggctcgcacttatgagtgcgcaggatttcgatcgcatcttcctgttcaatctgagcgatctcgacagccgagccggtccgcgcgatcagggctatggtctgctggatctgcaggccaaagccaagccggtatacaacgcgctggcgaatctgctgaaggttaccggtccgcgtctggagccgagtgatgccccgcgttttgaacaggctcccaaggatttgtacaacgtcacctgggtgcgtgaggacggcagccaggtatggatgttctggagtgccagtggcaagcagctccgccttccagctgtaacgcgtgctaccttgcacgatccgctcaccggtgaacggcgggaattgcagggcgctgagggtatcgacgtgccgcttaaatccagtctgcaactgttggtctggcgttag

SEQ ID NO: 11 - protein, 444 aa, Pseudomonas sp-62430
MWRRTLLFIPTFLLLGLILLVLPWSRQADAEVTTLKASGPLVWRDFLGVNAQFHFFEPDIYQAQMQQLSDLGLEWVRIAMHWAYLEPKRGQFNLVAFDPMVKAMQQHQLKPVGFLVGSAPFATTAPADSPYQDSFPPKDNALYSESLVRLAKRYDTFEAWQIWNEPNIFPFWRPKEDPQAYAKLLFQSASALRAHVPGKTVVAGGMAYYSNMPSHGGELMLQSLLQMGVAQQKLVMAYHPYTEKPEGASHKQQDYLQHSNFINGALRRHGIEQIWATEWGWSSYKGPREMQAIIGIDGQADYTLRRLALMSAQDFDRIFLFNLSDLDSRAGPRDQGYGLLDLQAKAKPVYNALANLLKVTGPRLEPSDAPRFEQAPKDLYNVTWVREDGSQVWMFWSASGKQLRLPAVTRATLHDPLTGERRELQGAEGIDVPLKSSLQLLVWR

SEQ ID NO: 12 - protein, 414 aa, Pseudomonas sp-62430
EVTTLKASGPLVWRDFLGVNAQFHFFEPDIYQAQMQQLSDLGLEWVRIAMHWAYLEPKRGQFNLVAFDPMVKAMQQHQLKPVGFLVGSAPFATTAPADSPYQDSFPPKDNALYSESLVRLAKRYDTFEAWQIWNEPNIFPFWRPKEDPQAYAKLLFQSASALRAHVPGKTVVAGGMAYYSNMPSHGGELMLQSLLQMGVAQQKLVMAYHPYTEKPEGASHKQQDYLQHSNFINGALRRHGIEQIWATEWGWSSYKGPREMQAIIGIDGQADYTLRRLALMSAQDFDRIFLFNLSDLDSRAGPRDQGYGLLDLQAKAKPVYNALANLLKVTGPRLEPSDAPRFEQAPKDLYNVTWVREDGSQVWMFWSASGKQLRLPAVTRATLHDPLTGERRELQGAEGIDVPLKSSLQLLVWR

SEQ ID NO: 13 - DNA, 1329 bp, Pseudomonas frederiksbergensis
  CDS (1)..(1326)
  sig_peptide (1)..(87)
  mat_peptide (88)..(1326)
atgacctcttgccgccgccctctcctgcctgtcgttgcggcgctgatgttcggcgctacgggtttgctcagccagccagccattgcagtgccgatcaatcttgcgtccgaccgcaccctggaatggaaagactatttgggggtgaatgcacactttttgtggttcaccccggcgcagtaccgcaagcagatcagcgcctatcagaagctggggctgcaatgggtgcgggtggacctgcactgggatcgcctggagccgaaggaagacgactatcagttgtcgacgcttgatgagctggacaagaccctgaccgccagcgggctcaagtcagtgttctatctggtcggctcggcgccgttcattacccgggcgccggtcggcgcgccgtttcaggatcaatacccgcccaaagaccccaaggtctatgccacgcgcatggccatgcttgcccaacgctaccccaacattgacgcctggcaggtgtggaacgagcagaacctgcccaacaactggcgcccgcaggtcgatcccgccgcctacggccaactgttgctggctacccatcaggcgctggaccaggtcgcgcccggtaaaacccaggtcatgggcggcatggcctactacagccagatgccgacgctgggcaaaaccctgatgttccaggccctcggcaaactcggcgtgcagagccttggcatggtcgcggcctatcacccttattccgtgacgccggaaactgacgagccgggcaaaaacgaagtactgctgcgcggcaagcaactcaacgacatgctgcacaacgccgggctgaaaaatgtttgggccaccgaatggggctggtccagttacgccggtccaagagaaatgcaggcgctgatcggcgttgatggccaggcggattacaccttgcggcgcctggcgctgatgagtacccaggactatcagcggatatttctcttcgcgctgtccgacctggatgatcgcgcctcggcccgcgaccagcactacggcctgcttgatctgaacggcgaaccaaaaccggtgtatcaggcattggcacgctttctcgacatcaccggcccacggctcaagcccggcaagacacccgtgctcgaaggcgcgcccgacagcttctacagcgtggcctggacccgcaatgacggcaaacaactgttgatgttctggagtgcagaaacgggcacgttgaaattgccggagattcatcaggccagcctttacgacccgctgaccggtacgcagcaaaacctcgacgcggcggacggcattacgcccggggtaaaaccgaccctgcagattctggtgtggtag

SEQ ID NO: 14 - protein, 442 aa, Pseudomonas frederiksbergensis
MTSCRRPLLPVVAALMFGATGLLSQPAIAVPINLASDRTLEWKDYLGVNAHFLWFTPAQYRKQISAYQKLGLQWVRVDLHWDRLEPKEDDYQLSTLDELDKTLTASGLKSVFYLVGSAPFITRAPVGAPFQDQYPPKDPKVYATRMAMLAQRYPNIDAWQVWNEQNLPNNWRPQVDPAAYGQLLLATHQALDQVAPGKTQVMGGMAYYSQMPTLGKTLMFQALGKLGVQSLGMVAAYHPYSVTPETDEPGKNEVLLRGKQLNDMLHNAGLKNVWATEWGWSSYAGPREMQALIGVDGQADYTLRRLALMSTQDYQRIFLFALSDLDDRASARDQHYGLLDLNGEPKPVYQALARFLDITGPRLKPGKTPVLEGAPDSFYSVAWTRNDGKQLLMFWSAETGTLKLPEIHQASLYDPLTGTQQNLDAADGITPGVKPTLQILVW

SEQ ID NO: 15 - protein, 413 aa, Pseudomonas frederiksbergensis
VPINLASDRTLEWKDYLGVNAHFLWFTPAQYRKQISAYQKLGLQWVRVDLHWDRLEPKEDDYQLSTLDELDKTLTASGLKSVFYLVGSAPFITRAPVGAPFQDQYPPKDPKVYATRMAMLAQRYPNIDAWQVWNEQNLPNNWRPQVDPAAYGQLLLATHQALDQVAPGKTQVMGGMAYYSQMPTLGKTLMFQALGKLGVQSLGMVAAYHPYSVTPETDEPGKNEVLLRGKQLNDMLHNAGLKNVWATEWGWSSYAGPREMQALIGVDGQADYTLRRLALMSTQDYQRIFLFALSDLDDRASARDQHYGLLDLNGEPKPVYQALARFLDITGPRLKPGKTPVLEGAPDSFYSVAWTRNDGKQLLMFWSAETGTLKLPEIHQASLYDPLTGTQQNLDAADGITPGVKPTLQILVW

SEQ ID NO: 16 - DNA, 1095 bp, Rhodococcus globerulus
  CDS (1)..(1092)
  sig_peptide (1)..(69)
  mat_peptide (70)..(1092)
gtgcgtcgacttcgattaccactcgtatgtgcactgctactgacgatcggatcgttaagcgcctgtgcacccaagccggtaacaacaacaacgacaagtgccccgcccgcaacctgcagttctgtcgggttgggtatcgcaggcggagcgccactgaattggctctcacaagccgatctggacaccgagttgagtgccatgaagaacgcaggcacgacatggctgcgcttcgacatcgactggtctgccgtcgaaccgaccaagggtcaacagaattgggcagcaactgatcgtgttgtcgatcgagcgagactacaaggattgagtctcgtcggaatcgttacctacacaccggcatgggcacgtgtggccggagcaaccgacactcatggttacccttctgacaccgcggcgtttgccaagttcgctcagcaagcagcgcaacgctattcaacgaggatcagcacttgggagatctggaacgagccgaacctcacacagttcttccgcccgaagcccaacgtcaacacctacgccgcgatactgaaggctgcgtcaaccagtatccgagcggtccaacccggggccaagatcctcaacggaggattagcgcccgcggtcgacaacggctccgacatatcaccggtcacctatctgaacgccctctacagcgccggcgctaaatcgtacttcgatgtgttctccatccacccgtacagttggcctgccttgccatccgacgcatccacctcgagttggaatactttctaccggattcgtttgatgcgcgacatcatggtgaagaatggtgacacgggaaagaaggtctgggcaacagaattcggcgctcctaccggatcgggatcaactgctgtcactccgcaactacaagccagcatcatctccgacggatttgcgcaggcacaggcactcggttacatcgaacgcatattcatctacagcatgcgtgatcgcggaaccaattcccgagacatcgagcagaatttcggtctggtgacgatcaactacacgccgaaacctgccctggacgcagtcaagaaggcaatcgggggttgcagcgcccccaagatctga

SEQ ID NO: 17 - protein, 364 aa, Rhodococcus globerulus
VRRLRLPLVCALLLTIGSLSACAPKPVTTTTTSAPPATCSSVGLGIAGGAPLNWLSQADLDTELSAMKNAGTTWLRFDIDWSAVEPTKGQQNWAATDRVVDRARLQGLSLVGIVTYTPAWARVAGATDTHGYPSDTAAFAKFAQQAAQRYSTRISTWEIWNEPNLTQFFRPKPNVNTYAAILKAASTSIRAVQPGAKILNGGLAPAVDNGSDISPVTYLNALYSAGAKSYFDVFSIHPYSWPALPSDASTSSWNTFYRIRLMRDIMVKNGDTGKKVWATEFGAPTGSGSTAVTPQLQASIISDGFAQAQALGYIERIFIYSMRDRGTNSRDIEQNFGLVTINYTPKPALDAVKKAIGGCSAPKI

SEQ ID NO: 18 - protein, 341 aa, Rhodococcus globerulus
PKPVTTTTTSAPPATCSSVGLGIAGGAPLNWLSQADLDTELSAMKNAGTTWLRFDIDWSAVEPTKGQQNWAATDRVVDRARLQGLSLVGIVTYTPAWARVAGATDTHGYPSDTAAFAKFAQQAAQRYSTRISTWEIWNEPNLTQFFRPKPNVNTYAAILKAASTSIRAVQPGAKILNGGLAPAVDNGSDISPVTYLNALYSAGAKSYFDVFSIHPYSWPALPSDASTSSWNTFYRIRLMRDIMVKNGDTGKKVWATEFGAPTGSGSTAVTPQLQASIISDGFAQAQALGYIERIFIYSMRDRGTNSRDIEQNFGLVTINYTPKPALDAVKKAIGGCSAPKI

SEQ ID NO: 19 - DNA, 1437 bp, Paenibacillus daejeonensis
  CDS (1)..(1434)
  sig_peptide (1)..(84)
  mat_peptide (85)..(1434)
atgcgacgaaacctgacgttattgatgcttgtcattgccttgctgctgcccggatttgcgggagcccctgagcaggcggaagcggcaccgaccaacaccaattttggcttcgctacagggtattccattctcaccatgagcaatacagacatgaatgcctggcttgacggtatggcggcgacgggagccgggtatatccggttcgacttcagctgggcctacatccagtcggggggttcgacatcctggaactggacgcaaaccgacagggttgtcgatgcggccttggccaaagggttcaagattctaccgattctctcgcatctgccgggatgggcaggctcgccctcaactatgaacgcctcgcattttcaacaattcgcctatcaggcggggctgcgctatattcccaagggcattacggactgggaactatggaatgaagccaatattcagggcttctctccggccaactacgtgaacaagattctgattccgggggccaatggtctgcggcaggcggcaagcgggcttaaccgtcaggtgacgatcgtctcaacaggtctggctccggctgcgacgaacggcacgcactggtccatgctggattacgtaacagggatctatgccaatggaggcaagaattacttcgatgcgctgggtgttcatccgtacacctggcctcagaatccaaccgtaatgacaaactggaactggctgcagaagacgccggagctctaccaggttatggtcaacaacggcgatagtcacaagaagctgtgggccacggagaacggctatcccacgagtacaaccaacggtgtaacggagcagcagcaggcccagtatatccaagccgcttatgaaatttgggactcgtacgccttcacagggggaccgtatttcatgtactcctacaaggatctgggcaccaatgtccaggatcccgaggatttcttcggcctggtgcggcacaacgggacgttgaagccggcgcatcagacggttgtcaatctcatcgcaggctcaacggccacgacgtacgtcaaaatccagaaccgctggaaggataaccagttcctctatgatgggggcacgcgcgtgcaatatggcaacggctcgggtgatgcgtacctgtgggctctggagtcctataacgggtacacccgcatccgcaacaaggcaaccggtgaatatattcatatcaagaacggtcagatgcaagtggacagtactgcgattgcagctacagatgttacgagccactggacgattgcgggctcctcggcaacgacctcagccaagtctattcgcagccgatccaacggcaactatctgaataatgaacagcagctgggctacgtgacctgcgaccgctctactgtacctcatgatacggcgtggtactcgcagcaatggtttctagtgccgcagtaa

SEQ ID NO: 20 - protein, 478 aa, Paenibacillus daejeonensis
MRRNLTLLMLVIALLLPGFAGAPEQAEAAPTNTNFGFATGYSILTMSNTDMNAWLDGMAATGAGYIRFDFSWAYIQSGGSTSWNWTQTDRVVDAALAKGFKILPILSHLPGWAGSPSTMNASHFQQFAYQAGLRYIPKGITDWELWNEANIQGFSPANYVNKILIPGANGLRQAASGLNRQVTIVSTGLAPAATNGTHWSMLDYVTGIYANGGKNYFDALGVHPYTWPQNPTVMTNWNWLQKTPELYQVMVNNGDSHKKLWATENGYPTSTTNGVTEQQQAQYIQAAYEIWDSYAFTGGPYFMYSYKDLGTNVQDPEDFFGLVRHNGTLKPAHQTVVNLIAGSTATTYVKIQNRWKDNQFLYDGGTRVQYGNGSGDAYLWALESYNGYTRIRNKATGEYIHIKNGQMQVDSTAIAATDVTSHWTIAGSSATTSAKSIRSRSNGNYLNNEQQLGYVTCDRSTVPHDTAWYSQQWFLVPQ

SEQ ID NO: 21 - protein, 450 aa, Paenibacillus daejeonensis
APTNTNFGFATGYSILTMSNTDMNAWLDGMAATGAGYIRFDFSWAYIQSGGSTSWNWTQTDRVVDAALAKGFKILPILSHLPGWAGSPSTMNASHFQQFAYQAGLRYIPKGITDWELWNEANIQGFSPANYVNKILIPGANGLRQAASGLNRQVTIVSTGLAPAATNGTHWSMLDYVTGIYANGGKNYFDALGVHPYTWPQNPTVMTNWNWLQKTPELYQVMVNNGDSHKKLWATENGYPTSTTNGVTEQQQAQYIQAAYEIWDSYAFTGGPYFMYSYKDLGTNVQDPEDFFGLVRHNGTLKPAHQTVVNLIAGSTATTYVKIQNRWKDNQFLYDGGTRVQYGNGSGDAYLWALESYNGYTRIRNKATGEYIHIKNGQMQVDSTAIAATDVTSHWTIAGSSATTSAKSIRSRSNGNYLNNEQQLGYVTCDRSTVPHDTAWYSQQWFLVPQ

SEQ ID NO: 22 - DNA, 1326 bp, Pseudomonas sp-62168
  CDS (1)..(1323)
  sig_peptide (1)..(87)
  mat_peptide (88)..(1323)
atgatgcgcaaaatgttttatctattgccgttggcggccctactggccggtgtcgtgctgctcaacccttggcagtcggccaaagctcagggcatgcaactaacagccacgcgcgatgtggtctggaaggacttccttggcgtcaatgcacactttctctggttccctcccgagcactaccgccaacagatgcagcagtggaaagccctgggcttggagtggacgcgcgttgacttgcactgggaccgtcacgagcctcgccaagggcaataccgtttgggtgagctagacggggtgatcggcgcgctcgccgacgaagacttaaagtcggtgttctatctggtgggttcggccccgcatgccacctcggccccggccaactcgccaacgccggatcaatacccgcccaaagacccggtcatgtttgccaagaccatggccatgcttgcccagcgttatgccacggtcgatgcctggcaggtgtggaacgagcccaatctgccgtcgttctggcgcccgcacgaagacgccgaaggctatggccgtctgctgctgcccagcgtgcaggccctgcgtcaggtcgtgccggagaagcccgtggtcatgggcggcatggcgtatttcagccaaatgcctgttaaaggcggtttgatgctggaggaactgggcaagttaggagtgcagcgactcggtacggtggtggcttatcacccgtattcacaagagccggaatacgacgagccgggcaccaacgactttattctgcgcacccagcaactcaatgccacgctgcgtaatgcgcaggtgccgggcatttgggcgactgaatggggttggtcgagctacaccggccccaaagagttgcaagagatcatcggcgagcaaggccaggccgattacgtgctgcgccgtttggccttgatgagcgcattggattttgaccggatcttcctgtttgccctggctgatctggacagtcgcgccaccgcgcgcgatcaacattacggcctgctcgatctgcaaggtcagcccaagccggtgtacaccgcgttgcagcgttttctgacgatcagtggcccgcgcttgcaaccgcagcagcccccacgcctgagtgttatgccggatgatctgtacagcgtcgcctggcagcgcgaagacggtcggcacctgtggatgttctggagcgccagcggtgccacgctgcaactgcccgagttaacccaggccgagttgcacgacccgctcaccgggcagcagcaaacactgaaaggtgccaacggcctgagcgtgcaagccaagcccggcctgcagatgttggtatggtaa

SEQ ID NO: 23 - protein, 441 aa, Pseudomonas sp-62168
MMRKMFYLLPLAALLAGVVLLNPWQSAKAQGMQLTATRDVVWKDFLGVNAHFLWFPPEHYRQQMQQWKALGLEWTRVDLHWDRHEPRQGQYRLGELDGVIGALADEDLKSVFYLVGSAPHATSAPANSPTPDQYPPKDPVMFAKTMAMLAQRYATVDAWQVWNEPNLPSFWRPHEDAEGYGRLLLPSVQALRQVVPEKPVVMGGMAYFSQMPVKGGLMLEELGKLGVQRLGTVVAYHPYSQEPEYDEPGTNDFILRTQQLNATLRNAQVPGIWATEWGWSSYTGPKELQEIIGEQGQADYVLRRLALMSALDFDRIFLFALADLDSRATARDQHYGLLDLQGQPKPVYTALQRFLTISGPRLQPQQPPRLSVMPDDLYSVAWQREDGRHLWMFWSASGATLQLPELTQAELHDPLTGQQQTLKGANGLSVQAKPGLQMLVW

SEQ ID NO: 24 - protein, 412 aa, Pseudomonas sp-62168
QGMQLTATRDVVWKDFLGVNAHFLWFPPEHYRQQMQQWKALGLEWTRVDLHWDRHEPRQGQYRLGELDGVIGALADEDLKSVFYLVGSAPHATSAPANSPTPDQYPPKDPVMFAKTMAMLAQRYATVDAWQVWNEPNLPSFWRPHEDAEGYGRLLLPSVQALRQVVPEKPVVMGGMAYFSQMPVKGGLMLEELGKLGVQRLGTVVAYHPYSQEPEYDEPGTNDFILRTQQLNATLRNAQVPGIWATEWGWSSYTGPKELQEIIGEQGQADYVLRRLALMSALDFDRIFLFALADLDSRATARDQHYGLLDLQGQPKPVYTALQRFLTISGPRLQPQQPPRLSVMPDDLYSVAWQREDGRHLWMFWSASGATLQLPELTQAELHDPLTGQQQTLKGANGLSVQAKPGLQMLVW

SEQ ID NO: 25 - DNA, 897 bp, Dyella sp-62115
  CDS (1)..(894)
  sig_peptide (1)..(66)
  mat_peptide (67)..(894)
gtgctgctcgtgctgcccctgctgctcgccgggtgcgtgcaggaggccgggtcggacaccgacgcggattcgggcgagaccgcgaccgccgctcccgccgaccagcccgcgaactggatctaccagctctccgggtacgccgacggcaaactcgacgcgctcgtcgcggccccccacgaggcggccgtgatcgacctcgcgcgcgacggcggcgaaggctacttcagcgccgacgagatcacctccctcgagaactccggcaagagcgtctacgcctacttcaccatgggctccatcgagacctaccggcccgaatacgacgccgtcgccgccaccgacatgatcctcaaccagtggggcgactggcccgacgagtacttcgtccagtactgggaccaggaatggtgggacctcgtcatgcagccccgcctcgaccaggccgccgccgccgggttcgacggcgtctacctcgacgtgcccaacgcctacgaggagatcgacctcgcgctcgtccccggggagacccgggaatcactggcgcagaagatggtcgacctcgtgatccgcgcgcaagagtacgccggggacgacctccagatcctcgtccagaactcccccgagctccgcgaataccccggctacctcgacgcgatcgacgggatcggcatcgaggagctgttcttcctcaacgccgacgagccctgcaccgaggactggtgcgccgagaacctcgacaacacccgcgcgatccgcgacgccgggaaactcgtcctcgccgtcgactacgcctccgaaccggccaacaccgccgccgcctgcgagcactacgccgaggagggattcgcgggagccgtcgccggagtcgacctcgacgcgatctacgagccctgcccctga

SEQ ID NO: 26 - protein, 298 aa, Dyella sp-62115
VLLVLPLLLAGCVQEAGSDTDADSGETATAAPADQPANWIYQLSGYADGKLDALVAAPHEAAVIDLARDGGEGYFSADEITSLENSGKSVYAYFTMGSIETYRPEYDAVAATDMILNQWGDWPDEYFVQYWDQEWWDLVMQPRLDQAAAAGFDGVYLDVPNAYEEIDLALVPGETRESLAQKMVDLVIRAQEYAGDDLQILVQNSPELREYPGYLDAIDGIGIEELFFLNADEPCTEDWCAENLDNTRAIRDAGKLVLAVDYASEPANTAAACEHYAEEGFAGAVAGVDLDAIYEPCP

SEQ ID NO: 27 - protein, 276 aa, Dyella sp-62115
DSGETATAAPADQPANWIYQLSGYADGKLDALVAAPHEAAVIDLARDGGEGYFSADEITSLENSGKSVYAYFTMGSIETYRPEYDAVAATDMILNQWGDWPDEYFVQYWDQEWWDLVMQPRLDQAAAAGFDGVYLDVPNAYEEIDLALVPGETRESLAQKMVDLVIRAQEYAGDDLQILVQNSPELREYPGYLDAIDGIGIEELFFLNADEPCTEDWCAENLDNTRAIRDAGKLVLAVDYASEPANTAAACEHYAEEGFAGAVAGVDLDAIYEPCP

SEQ ID NO: 28 - DNA, 1308 bp, Pseudomonas fulva
  CDS (1)..(1305)
  sig_peptide (1)..(66)
  mat_peptide (67)..(1305)
atgcgcaaactgctaccttgcctggccgtgctgctgcagggcttcttcactccgggcgcgcaggcggccaacgaacctttcatcatcggcaccgcaacccatgtgatggatggttcaccgcagcttgcgcaccagttccagctggccagcgaagcgggtatcggctccttgcgcgaagacgcctactgggcccgcgtggaactgcagccgggcaccctgcaggtacctgccagctggcgcgcttaccagaaggagcgcgaggccaggaagctgggcaacgtggtggtgctcgattacggcaaccagttctatgacaacaacgcgctgccacgttcgcccatggtcagcaccgcctttgccaactacgtggatttcgtgacccgggcgttggccggcacggtcaacttctacgaggtctggaatgaatgggaccaggccgggcccggcgaccgggccgtcagtgatgactatgccagcctggtcaaactcacccgccagcagattcaacgcaatgacccgaaggcgaaggtgctggccggtgccatcaccagcgacgggctgaacaagggcttcgctgaccgcctggtccaggccggcctggccgagcaggtcgacggcctgtcattgcacccctatgtgcactgcgccggcaaacagggcaagacaccggagagttggatcaagtggctgtccagcatcgaccagcgcctgacgcgcctggcgggaaagccggtaccgctgtacctcacggaaatgagctggcccacctcgagcgaaaaaacctgcggggtggacgagcccacgcaggccaagttcctggccagggcgtacttcctggccaagacacgccccaacatcaagggcatgtggtggtacgacctggtggatgacggcgtggacccggacgagcgtgaacaccatttcggcctgctcaggccgggcctggagcccaagccggcctaccgggtgctcaaggccatcgcgccgtttctggcgcagtaccaatacgacagcctgaagagcctgcaaaccgacgagttgtacctgctcaatttcaccaagggcgatgagcaggtgctggtggcctgggcggtgggcgacccccgccaggtgaagatcgaggccaacggccgccagcaggggccagtacagatggtcgacacccatcaccccgaacgcggccgcaccgccaccggccaatggcaatgccccaaggctgaagaagaacactgcaccacggtgatcaccctggacgattttccccgaatcatcagcctgggcgacgccagctggctattcacccgctga

SEQ ID NO: 29 - protein, 435 aa, Pseudomonas fulva
MRKLLPCLAVLLQGFFTPGAQAANEPFIIGTATHVMDGSPQLAHQFQLASEAGIGSLREDAYWARVELQPGTLQVPASWRAYQKEREARKLGNVVVLDYGNQFYDNNALPRSPMVSTAFANYVDFVTRALAGTVNFYEVWNEWDQAGPGDRAVSDDYASLVKLTRQQIQRNDPKAKVLAGAITSDGLNKGFADRLVQAGLAEQVDGLSLHPYVHCAGKQGKTPESWIKWLSSIDQRLTRLAGKPVPLYLTEMSWPTSSEKTCGVDEPTQAKFLARAYFLAKTRPNIKGMWWYDLVDDGVDPDEREHHFGLLRPGLEPKPAYRVLKAIAPFLAQYQYDSLKSLQTDELYLLNFTKGDEQVLVAWAVGDPRQVKIEANGRQQGPVQMVDTHHPERGRTATGQWQCPKAEEEHCTTVITLDDFPRIISLGDASWLFTR

SEQ ID NO: 30 - protein, 413 aa, Pseudomonas fulva
ANEPFIIGTATHVMDGSPQLAHQFQLASEAGIGSLREDAYWARVELQPGTLQVPASWRAYQKEREARKLGNVVVLDYGNQFYDNNALPRSPMVSTAFANYVDFVTRALAGTVNFYEVWNEWDQAGPGDRAVSDDYASLVKLTRQQIQRNDPKAKVLAGAITSDGLNKGFADRLVQAGLAEQVDGLSLHPYVHCAGKQGKTPESWIKWLSSIDQRLTRLAGKPVPLYLTEMSWPTSSEKTCGVDEPTQAKFLARAYFLAKTRPNIKGMWWYDLVDDGVDPDEREHHFGLLRPGLEPKPAYRVLKAIAPFLAQYQYDSLKSLQTDELYLLNFTKGDEQVLVAWAVGDPRQVKIEANGRQQGPVQMVDTHHPERGRTATGQWQCPKAEEEHCTTVITLDDFPRIISLGDASWLFTR

SEQ ID NO: 31 - DNA, 1038 bp, Rahnella sp-62576
  CDS (1)..(1035)
  sig_peptide (1)..(66)
  mat_peptide (67)..(1035)
atgagtaaagtggttattttcatgaaaattttatgtttaatgattttttgctacccattctacggaatgtgcacaattattggtgtaggtactcattttcagggatatcgtggagacagcgagaactatttagtaaagattaaaagtctgggtttcacttcgttcagagaagattacccgtggtcaaatgtcgagaaaactaaaggaagttttgctgtaagtgacagcatcaggaaaaaagactcggcattcctaaaggcaaagggaaacggtctggaacctgtactcattcttgattatggaaataaattctacaatgacggtgattatcctagaaatgaagaatcaataaatgcatttgtaaaatatgcaacctggactgcaacaagattcaaagggaaagtaaaatattatgaggtttggaatgaatggactatcggcactggtatgacaaagtatcgcaagaacattccttctgcagaaatttattttaatctggttaaagcgacgagcgaggcgataaaaaaaatagaccccgatgcaatcattttagccggcggttttaatcctttagagcagagagctaagtttatcgacgtcactgatacagtctggtttagccagttgctaaaactggggattttaaattatgcagacgggatctcgattcacacctattcctaccttaatggaaggcgctcattaagaacggtggaagggaatttagattatctggatagcttccatgctgccagtgaaaaaatagcaggaaaaggtgttccattttatattactgaaatcggtgtcaccaactacactggccctggcggcatgaaagaagatgaggccgccaattatattaaagaatatatcaagagtgcaataacgcggaattacatcaaaggggtatggatttatgaccttatcgatgatggtaaggacaaaagtaagagagacttcaactttggtttactcaataacgatttatccccgaagcaggccgcaccggttgtttctcagtttcttaatggtaagtaa

SEQ ID NO: 32 - protein, 345 aa, Rahnella sp-62576
MSKVVIFMKILCLMIFCYPFYGMCTIIGVGTHFQGYRGDSENYLVKIKSLGFTSFREDYPWSNVEKTKGSFAVSDSIRKKDSAFLKAKGNGLEPVLILDYGNKFYNDGDYPRNEESINAFVKYATWTATRFKGKVKYYEVWNEWTIGTGMTKYRKNIPSAEIYFNLVKATSEAIKKIDPDAIILAGGFNPLEQRAKFIDVTDTVWFSQLLKLGILNYADGISIHTYSYLNGRRSLRTVEGNLDYLDSFHAASEKIAGKGVPFYITEIGVTNYTGPGGMKEDEAANYIKEYIKSAITRNYIKGVWIYDLIDDGKDKSKRDFNFGLLNNDLSPKQAAPVVSQFLNGK

SEQ ID NO: 33 - protein, 323 aa, Rahnella sp-62576
MCTIIGVGTHFQGYRGDSENYLVKIKSLGFTSFREDYPWSNVEKTKGSFAVSDSIRKKDSAFLKAKGNGLEPVLILDYGNKFYNDGDYPRNEESINAFVKYATWTATRFKGKVKYYEVWNEWTIGTGMTKYRKNIPSAEIYFNLVKATSEAIKKIDPDAIILAGGFNPLEQRAKFIDVTDTVWFSQLLKLGILNYADGISIHTYSYLNGRRSLRTVEGNLDYLDSFHAASEKIAGKGVPFYITEIGVTNYTGPGGMKEDEAANYIKEYIKSAITRNYIKGVWIYDLIDDGKDKSKRDFNFGLLNNDLSPKQAAPVVSQFLNGK

SEQ ID NO: 34 - DNA, 1236 bp, Pseudomonas aeruginosa
  CDS (1)..(1236)
  mat_peptide (1)..(1236)
gaaattcaagtacttaaagcacctcgtgctgttgtttggaaagacttccttggtgtaaacgcacaattcttatggttttctccagaacgctacaataagcaaattgatcgccttcaagatttaggtcttgagtgggtacgcttagatcttcactgggatcgtcttgaaacggctgaagaccagtaccaattggcctctttagaccaattggttaaagatcttgaagctcgtcagcttaagtctgtattctatcttgtaggatctgctcgcttcattactacagctccgttttacagcccatttcaagatcaatacccaccacgcgatcctgaagttttcgctcgtcgtatggccatgttatcacaacgttacccatctgttgcagcgtggcaggtatggaatgaaccgaaccttattggtttttggcgtccaaaagctgatcctgaaggctacgctaaacttcttcaagcgtctactatcgctttacgtatggttgatccagaaaaaccagtagtttcagctggtatggctttcttttctgagatgcctgatggccgtacgatgtttgacgctcttggtcaccttggcgtagaatctcttggcacaatcgcaacgtaccacccttacacgcaacttcctgaaggcaactatccttggaacttagactttgtatctcacgcgaaccagatcaatcgcgctcttcgtaacgcaggcgttccagcaatctggtctactgaatggggctggagcgcatacaaaggtccaaaagagttacaagacattatcggagtagaaggtcaggctgattacgttttgcgtcgtttggcccttatgtctgctcttgactatgatcgcattttccttttcactttatctgatcttgatcaacgtgcgtcagttcgtgatcgcgattacggattacttgacttagatgcaaatcctaaaccagtttaccttgcattgcaacgcttcttgaaagtaactggtccaaagcttcgcccagctgaccctccggtaactgaagaccttccagacggcagcttttcaattggctggactcgcgaagacggtcgtaatgtatggttattctggtctgcacgtggtggtaacgttcgtcttcctaagcttaaagaggctacgcttcatgatcctttgtctggaaaagttacgccattatctggcagcgacggtttggaagtacctgttaagtcttctttacaaatgttagtttgggaa

SEQ ID NO: 35 - protein, 412 aa, Pseudomonas aeruginosa
EIQVLKAPRAVVWKDFLGVNAQFLWFSPERYNKQIDRLQDLGLEWVRLDLHWDRLETAEDQYQLASLDQLVKDLEARQLKSVFYLVGSARFITTAPFYSPFQDQYPPRDPEVFARRMAMLSQRYPSVAAWQVWNEPNLIGFWRPKADPEGYAKLLQASTIALRMVDPEKPVVSAGMAFFSEMPDGRTMFDALGHLGVESLGTIATYHPYTQLPEGNYPWNLDFVSHANQINRALRNAGVPAIWSTEWGWSAYKGPKELQDIIGVEGQADYVLRRLALMSALDYDRIFLFTLSDLDQRASVRDRDYGLLDLDANPKPVYLALQRFLKVTGPKLRPADPPVTEDLPDGSFSIGWTREDGRNVWLFWSARGGNVRLPKLKEATLHDPLSGKVTPLSGSDGLEVPVKSSLQMLVWE

SEQ ID NO: 36 - protein, 412 aa, Pseudomonas aeruginosa; identical to SEQ ID NO: 35

SEQ ID NO: 37 - protein, 5 aa, Artificial
  Motif
  MISC_FEATURE (1)..(1): Xaa = A (Ala) or G (Gly) or S (Ser)
  MISC_FEATURE (2)..(2): Xaa = any amino acid
XXHPY

SEQ ID NO: 38 - protein, 6 aa, Artificial
  Motif
  MISC_FEATURE (1)..(1): Xaa = I (Ile) or V (Val) or L (Leu) or F (Phe) or M (Met)
  MISC_FEATURE (2)..(2): Xaa = Y(Try) or W (Trp) or F (Phe)
  MISC_FEATURE (3)..(3): Xaa = any amino acid
  MISC_FEATURE (4)..(4): Xaa = T (Thr) or S (Ser)
  misc_feature (5)..(5): Xaa can be any naturally occurring amino acid
  MISC_FEATURE (6)..(6): Xaa = any amino acid
XXXEXG

SEQ ID NO: 39 - protein, 10 aa, Artificial
  Motif
  MISC_FEATURE (1)..(1): Xaa = D (Asp) or G (Gly) or I (Ile) or V (Val)
  MISC_FEATURE (2)..(2): Xaa = any amino acid
  MISC_FEATURE (3)..(3): Xaa = any amino acid
  MISC_FEATURE (4)..(4): Xaa = any amino acid
  MISC_FEATURE (5)..(5): Xaa = E (Glu) or Q (Gln)
  MISC_FEATURE (6)..(6): Xaa = I (Ile) or L (Leu) or V (Val)
  MISC_FEATURE (10)..(10): Xaa = P (Pro) or Q (Gln) or W (Trp) or F (Phe)
XXXXXXWNEX

SEQ ID NO: 40 - protein, 5 aa, Artifical
  MISC_FEATURE (1)..(1): Xaa = A (Ala) or N (Asn) or T (Thr) or V (Val)
XWQVW

SEQ ID NO: 41 - protein, 27 aa, Artificial
  Signal peptide
MKKPLGKIVASTALLISVAFSSSIASA

SEQ ID NO: 42 - protein, 8 aa, Artificial
  His tag
HHHHHHPR